SEQUENCE LISTING (4 sequences)

SEQ ID NO: 1 - DNA, 20 bp, Artificial Sequence
  Synthetic oligo 1
gccttacgcttcctcccgcg

SEQ ID NO: 2 - DNA, 20 bp, Artificial Sequence
  Synthetic oligo 2
ctcggctacggcgaccaccg

SEQ ID NO: 3 - DNA, 21 bp, Artificial Sequence
  Synthetic oligo 1
gtaaggcgggggccgcgccta

SEQ ID NO: 4 - DNA, 21 bp, Artificial Sequence
  Synthetic oligo 2
gcgccttacgctttcttccgt

(19) United States
(12) Patent Application Publication
(10) Pub. No.: US 2024/0118263 A1
(43) Pub. Date: Apr. 11, 2024

(54) TARGET FOR SCREENING ANTI-TUMOR DRUG, USE THEREOF AND SCREENING METHOD THEREFOR

(71) Applicant: SHENYANG FUYANG PHARMACEUTICAL TECHNOLOGY CO., LTD., Shenyang, Liaoning (CN)

(72) Inventors: Enhong JIANG, Shenyang, Liaoning (CN); Weiqing HE, Shenyang, Liaoning (CN); Mingyu XIA, Shenyang, Liaoning (CN); Dong WANG, Shenyang, Liaoning (CN); Xundong JIANG, Shenyang, Liaoning (CN); Xiaofeng ZHAO, Shenyang, Liaoning (CN)

(73) Assignee: SHENYANG FUYANG PHARMACEUTICAL TECHNOLOGY CO., LTD., Shenyang, Liaoning (CN)

(21) Appl. No.: 17/768,149

(22) PCT Filed: Oct. 10, 2020
(86) PCT No.: PCT/CN2020/120080

§ 371 Date: Apr. 11, 2022

(30) Foreign Application Priority Data
Oct. 11, 2019 (CN) 201910964581.3
Mar. 11, 2020 (CN) 202010166309.3

(51) Int. Cl. G01N 33/50; C07K 14/47; G01N 33/574; G01N 33/68

(52) U.S. Cl. CPC G01N 33/5011; C07K 14/47; G01N 33/574; G01N 33/68; G01N 2333/902

(57) ABSTRACT

Disclosed are target for screening anti-tumor drug, application use thereof and screening method therefor. Disclosed are a target for screening a drug for treating and/or preventing tumors, the target for screening the drug for treating and/or preventing tumors comprise selenoprotein; and a target for screening a drug for preventing tumor metastasis, the target for screening the drug for preventing the tumor metastasis comprising selenoprotein. Also disclosed is a method for screening a drug for treating and/or preventing tumors or a drug for preventing tumor metastasis, the method comprising: interacting a candidate drug with the selenoprotein; and screening the drug for treating and/or preventing tumors or the drug for preventing tumor metastasis according to an affinity between the candidate drug and the selenoprotein; and the candidate drug with a high affinity with the selenoprotein is taken as a candidate primarily screened drug.

### TECHNICAL FIELD

The disclosure belongs to the field of medicines, and particularly relates to a target for screening anti-tumor drug, use thereof and screening method therefor.

### BACKGROUND

Traditional anti-cancer regimens typically utilize cell killing functions, such as radiation, chemotherapy, and recent T-cell activation or weaponization. This universal method will cause off-target toxicity and other adverse side effects, such as immune response disorders. Thus, an alternative approach is required in which a mechanism of the anti-cancer action is less dependent on the cell killing action and downstream consequences thereof.

A selenoprotein is a protein in which selenium is covalently bound to prokaryotic or eukaryotic cells. A selenoprotein family is a main component of selenium functioning, storage, and exerting its antioxidant activity in the body. So far, more than 30 selenoproteins have been found in prokaryotic and eukaryotic cells, mainly a glutathione peroxidase (GPx) selenoenzyme family, an iodothyronine deiodinase family, a thioredoxin reductase family and other selenoproteins with unknown functions: SPS2, SelP, SelW, PEs, SelS, SelH, SelX, SelK, SelM, SelN, SelO, SelR, SelS, SelT, SelV, SelX, SelY, SelZ and the like. Selenium exerts its physiological function through selenoprotein. Selenium in the selenoprotein exists in the form of selenocysteine (SeCys), and the SeCys is mostly located at the active center of the protein and plays an important role in the structure and function of the protein.

Selenium has many biochemical functions, and most importantly, the selenium has an anti-oxidation effect. The anti-oxidation effect of selenium mainly includes the following aspects: 1. decomposing lipid peroxides; 2. scavenging lipid peroxidation free radical intermediates; 3. catalyzing a reaction with a sulfhydryl compound as a protective agent; 4. scavenging or converting the hydrated free radicals into stable compounds before hydrated free radicals destroy the living substances; and 5. repairing molecular damage of sulfur compounds caused by the hydrated free radicals.

Selenium participates in the formation of glutathione peroxidase (GSH-Px) in an organism in the form of selenocysteine (SeCys). Glutathione peroxidase catalyzes the conversion of GSH (reduced glutathione) to GSSG (oxidized glutathione) through a reaction. On one hand, peroxide with toxicity is converted into a hydroxyl compound without toxicity. On the other hand, H₂O₂ is decomposed at the same time, damage of peroxide to cell membranes is reduced, the integrity of cell membrane structures is guaranteed, and the normal functions of the cell membranes are maintained.

A selenoprotein H is a recently discovered functional mammalian protein with a protein size of 14 kDa. Through specific sequence and structure analysis, the selenoprotein H is determined as thioredoxin with a folded structure, wherein a conservative basic structure CXXU (U represents selenocysteine) corresponds to a CXXC structure of the thioredoxin. These data indicate the redox function of the SelH. The recombinant SelH shows the obvious activity of glutathione peroxidase. In addition, the SelH has a sequence-conserved RKRK nuclear localization signal sequence at the N-terminal, and experiments prove that the SelH is also specifically distributed in a nucleolus. Northem hybridization analysis reveals that the mRNA expression level of SelH in various tissues of mice is low, but the expression level is increased in the early stage of embryonic development. In addition, researches prove that the SelH is related to cancers, and the mRNA expression quantity of the SelH in human prostate cancer LNCaP and mouse lung cancer LLC cells is extremely high.

In order to further verify the function of the SelH and the possibility of the SelH serving as a novel drug target, realize heterologous expression of the SelH, and obtain a high-purity protein, it is very necessary to carry out drug screening and structural biology exploration.

In view of this, the present disclosure is specifically proposed.

### SUMMARY

The technical problem to be solved by the disclosure is to overcome the defects in the prior art and provide a target for screening anti-tumor drug, use thereof and screening method therefor. The disclosure provides a novel method and target for screening an anti-tumor drug.

In order to solve the above technical problem, a basic conception of the technical solution adopted by the present disclosure is as follows.

In a first aspect, the disclosure provides a target for screening a drug for treating and/or preventing tumors, and the target for screening the drug for treating and/or preventing tumors includes selenoprotein; preferably, the selenoprotein is selenoprotein H.

The selenoprotein H is a nucleolar protein containing a selenocysteine residue at an active site, and plays a key role in protecting DNA from oxidative damage and relieving genomic instability. Drugs can induce accumulation of reactive oxygen species (ROS) by inhibiting the selenoprotein H (SelH) in a cell nucleus, especially the SelH in a nucleolus. In addition, through a JNK2/TIF1A pathway in the nucleolus, drugs such as carrimycin and isovalerylspiramycin I can promote increase of DNA damage and decrease of RNA polymerase (Pol) I transcription, so that proliferation and apoptosis of cancer cells are inhibited. The action of these molecular levels results in inhibition of tumors or inhibition of tumor metastasis. Therefore, the selenoprotein, especially the selenoprotein H, can be used as a target for screening drugs for treating and/or preventing tumors and drugs for preventing tumor metastasis.

In a second aspect, the disclosure provides a target for screening a drug for preventing tumor metastasis, and the target for screening the drug for preventing the tumor metastasis includes selenoprotein; preferably, the selenoprotein is selenoprotein H.

Tumor metastasis is a process of spreading cancer cells from one organ to another one or more non-adjacent organs. More specifically, during metastasis, subpopulations of cancer cells in a primary lesion adapt to selective stress, causing these cells to spread, invade and propagate in an unfavourable non-natural environment. The drugs for preventing tumor metastasis screened by using selenoprotein are used to destroy the metastasis process in the disclosure, so that the risk of cancer cell spreading in patients is reduced.

In a third aspect, the disclosure provides an application of the selenoprotein as a drug action target in screening drugs for treating and/or preventing tumors and drugs for preventing tumor metastasis; preferably, the disclosure provides an application of the selenoprotein H as a drug action target in screening drugs for treating and/or preventing tumors and drugs for preventing tumor metastasis in vitro.

The selenoprotein H binds to the acyl of a drug, and the acyl is acyl of which the carbon atom number is greater than or equal to 3, and preferably, the acyl is acyl of non-linear carbon; more preferably, the acyl is isovaleryl of isovalerylspiramycin.

Further, the selenoprotein interacts with an isovaleryl, and/or an isopentenyl, and/or an isopentenyl-like group of a candidate drug.

In a fourth aspect, the disclosure provides a method for screening drugs for treating and/or preventing tumors or drugs for preventing tumor metastasis, including: screening drugs by taking selenoprotein as a drug action target; preferably, the selenoprotein is human selenoprotein; more preferably, the selenoprotein is selenoprotein H.

According to a further solution, the method includes the following steps of:
(1) interacting candidate drugs with selenoprotein; and
(2) screening the drugs for treating and/or preventing tumors and the drugs for preventing tumor metastasis according to the affinity between the candidate drugs and the selenoprotein.

According to a further solution, the candidate drugs with high affinity with the selenoprotein are taken as candidate primarily screened drugs.

The binding affinity refers to a strength of mutual binding between a single biomolecule (e.g., protein or DNA) and its ligand/binding partner (e.g., a drug or inhibitor). The binding affinity is generally measured and reported by an equilibrium dissociation constant (KD) that is used to evaluate the intensity of the bimolecular interaction and to sort such intensities. The smaller a KD value, the greater the binding affinity of a ligand to its target.

According to a further solution, the candidate primarily screened drugs is selected from a group consisting of a compound with an isopentenyl group, a compound with an isopentenyl-like group, a compound with an isovaleryl group, a macrolide compound and a cyclic peptide compound.

According to a further solution, the candidate primarily screened drugs is selected from a group consisting of a coumarin compound with the isopentenyl group, and/or the isovaleryl group, and/or the isopentenyl-like group, or a triterpenoid compound with the isopentenyl group, and/or the isovaleryl group, and/or the isopentenyl-like group, or a flavonoid compound with the isopentenyl group, and/or the isovaleryl group, and/or the isopentenyl-like group, or a macrolide compound with the isopentenyl group, and/or the isovaleryl group, and/or the isopentenyl-like group, or a shikonin compound with the isopentenyl group, and/or the isovaleryl group, and/or the isopentenyl-like group.

According to a further solution, the coumarin compound or the triterpenoid compound with the isopentenyl group, and/or the isovaleryl group, and/or the isopentenyl-like group include but are not limited to aurapten (shown in a formula I), iso-imperatorin (shown in a formula II), protopanoxadiol (shown in a formula III), decursin (shown in a formula IV), osthol (shown in a formula V), notoginsenoside R1 (shown in a formula VI), and shionon (shown in a formula VII), and structural formulae of the compounds are shown as follows:

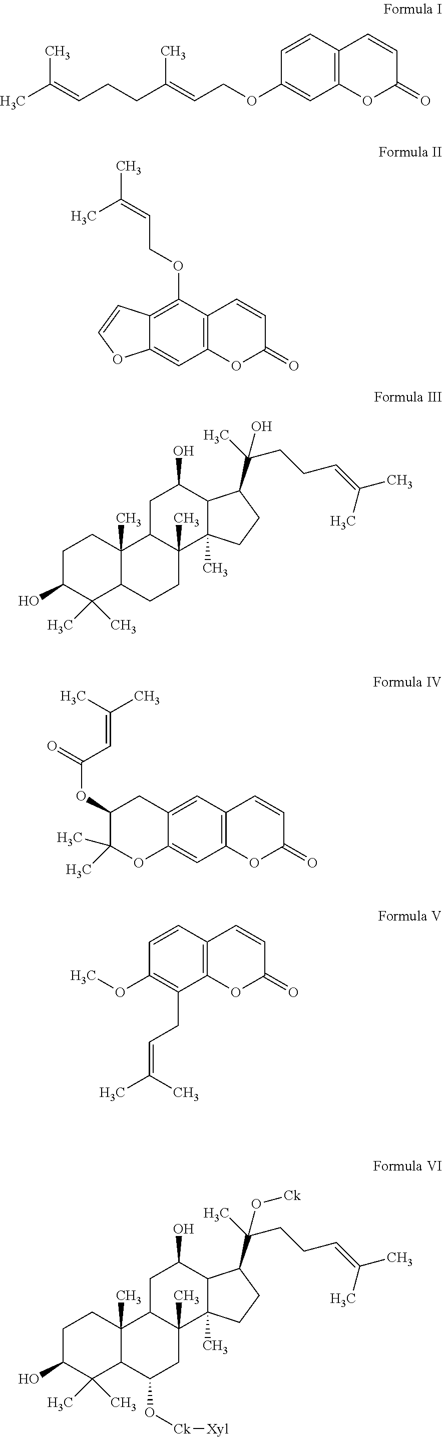

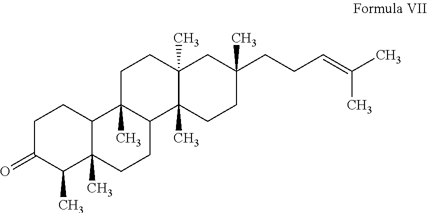

According to a further solution, isopentenyl substituted flavonoid or shikonin compounds include but are not limited to the following several compounds: acetyl shikonin (shown in a formula VIII), anthraquinone (shown in a formula IX), isoxanthohunol (shown in a formula X), α-mangostin (shown in a formula XI), morusin (shown in a formula XII) and shikonin (shown in a formula XIII), and structural formulas of the compounds are shown as follows:

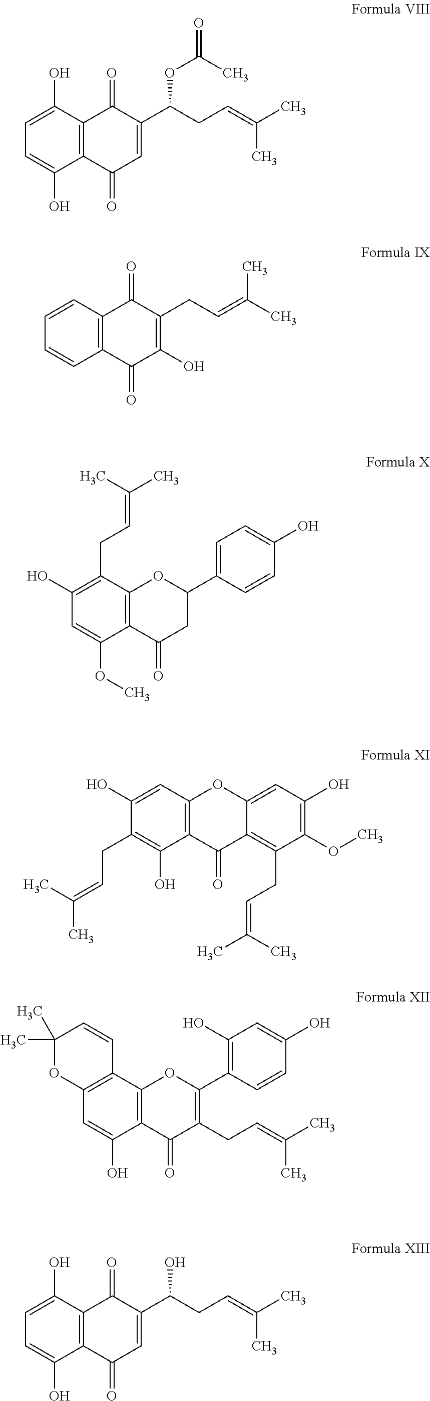

According to a further solution, the macrolide and cyclic peptide compounds include but are not limited to carrimycin, isovalerylspiramycin I, isovalerylspiramycin II, isovalerylspiramycin III, spiramycin, carbomycin, azithromycin, erythromycin and thiostrepton.

A pharmaceutical composition containing any one or more of 4″-isovalerylspiramycin I, II and III (collectively referred to as “isovalerylspiramycin” or “ISP”) act on cancer cells to trigger genomic instability, thereby inhibiting proliferation by promoting cell cycle arrest, and reducing adverse side effects.

According to a further solution, the tumors include solid tumors and non-solid tumors. Wherein the neoplastic disease may be characterized by nucleolar hypertrophy, may relate to tumors lacking DNA damage repair, and/or may relate to cancers exhibiting accelerated rRNA synthesis.

Preferably, the solid tumors include benign solid tumors and malignant solid tumors, and the non-solid tumors include lymphoma or leukemia; preferably, the malignant solid tumors include breast cancer, liver cancer, lung cancer, kidney cancer, brain tumor, cervical cancer, prostate cancer, lymph cancer, pancreatic cancer, esophageal cancer, gastric cancer, colon cancer, thyroid cancer, bladder cancer or malignant skin tumor; preferably, the malignant skin tumor includes melanoma.

According to a further solution, the tumors are selected from diffuse large B-cell lymphoma, acute myeloid leukemia, pancreatic adenocarcinoma, thyroid cancer, thymoma, uterine endometrial cancer, uterine carcinosarcoma and uveal melanoma.

According to a further solution, the method further includes the following steps: carrying out an in-vitro test on the candidate primarily screened drugs, and further screening out drugs with an inhibiting effect on tumor cells and/or a preventing effect on tumor metastasis.

After the above technical solution is adopted, compared with the prior art, the present disclosure has the following beneficial effects:

the disclosure provides a novel action target for screening the drugs for treating and/or preventing tumors and the drugs for preventing tumor metastasis, and a novel screening method for screening the drugs for treating and/or preventing tumors and the drugs for preventing tumor metastasis, which proves that the selenoprotein, especially the selenoprotein H, can be used as the screening action target. The compounds capable of inhibiting tumor cells and tumor cell metastasis in vitro can be preliminarily screened by detecting the affinity between the candidate compounds and the selenoprotein, so that the drugs for treating and/or preventing tumors and the drugs for preventing tumor metastasis can be rapidly and efficiently screened.

Specific embodiments of the present disclosure will be further described below in detail in combination with the accompanying drawings.

### BRIEF DESCRIPTION OF THE DRAWINGS

The drawings serving as one part of the present disclosure are intended to provide a further understanding for the present disclosure. Schematic embodiments of the present disclosure and the descriptions thereof are intended to explain the present disclosure, rather than an improper limitation of the present disclosure. Obviously, the drawings described below are merely some embodiments. Under the precondition of not paying any inventive labor, those of ordinary skill in the art can further obtain other drawings according to these drawings. In the drawings:

FIG. 1A-G are a surface plasmon resonance (SPR) detection result of the affinity between compounds and SelH in Embodiment 2 of the disclosure;

FIG. 2 is the correlation analysis between the affinity of the compounds and the SelH, and the cytotoxic effect on 4T1 cells in Embodiment 2 of the disclosure;

FIG. 3 is the correlation analysis between the affinity of the compounds and the SelH, and the cytotoxic effect on B16-BL6 cells in Embodiment 2 of the disclosure;

FIG. 4 is the correlation analysis between the affinity of the compounds and the SelH, and the cytotoxic effect on A549 cells in Embodiment 2 of the disclosure;

FIG. 5A-F are a surface plasmon resonance (SPR) detection result of the affinity between compounds and SelH in Embodiment 3 of the disclosure;

FIG. 6 is the correlation analysis between the affinity of the compounds and the SelH, and the cytotoxic effect on B16-BL6 cells in Embodiment 3 of the disclosure;

FIG. 7A-7G is a surface plasmon resonance (SPR) detection result of the affinity between compounds and SelH in Embodiment 4 of the disclosure; and

FIG. 8 is the correlation analysis between the affinity of the compounds and the SelH, and the cytotoxic effect on A549 cells in Embodiment 4 of the disclosure;

FIG. 9 is a combined graphic and tabular presentation of ISP I's cytotoxicity for glioblastoma cells. FIG. 9A is a line graph of CCK8 assay data that delineate ISP I dose-response curves in relation to five glioblastoma cell lines, T98G, U118, A172, LN229 and U251, for a 48-hour period.

FIG. 9B is a table that lists IC50 values calculated for each glioblastoma cell line. FIG. 9C depicts flow cytometry data that evidence the cell-cycle effect of ISP I treatment on LN229 and U251 cells.

FIG. 9D presents in bar graph format a cell-cycle analysis showing G0/G1 arrest in ISP I-treated cells. FIG. 9E depicts flow cytometry data from an apoptosis (Annexin-V stain) analysis of LN229 and U251 cells with ISP I treatment. FIG. 9F presents in bar graph format results of apoptosis analysis (4 independent wells) of ISP I-treated LN229 and U251 cells. All data are shown as mean±s.e.m. P value: *p<0.05; **p<0.01; ***p<0.001;

FIG. 10 is a combined graphic, schematic and tabular presentation of the cytotoxicity of ISP I in renal cell carcinoma (RCC) cells. FIG. 10A is a line graph of dose-response CCK8 data for ISP I for RCC cell lines ACHN, UM-RC-2, RCC4 and 786-O for 48 hours. FIG. 10B is a table of IC50 values calculated for ISP I for each of the same cell lines. FIG. 10C depicts results from cell-cycle analysis of 786-O and RCC4 cells treated with ISP I. FIG. 10D presents bar graphs of cell-cycle progression data, showing clear G0/G1 arrest in ISP I-treated RCC cells. FIG. 10E portrays results from an Annexin-V apoptosis analysis of 786-O and RCC4 cells treated with ISP I. FIG. 10F summarizes in bar graph format results of the apoptosis analysis (four independent wells). All data are shown as mean±s.e.m. P value: *p<0.05; **p<0.01; ***p<0.001;

FIG. 11 is combined schematic and graphic presentation of ISP I's targeting to SELH in glioblastoma cell lines. FIG. 11A is a schematic summary of a Drug Affinity Responsive Target Stability (DARTS) assay. FIG. 11B presents western blotting results for SELH expression in LN229 cells, showing that ISP I-protected SELH was observed along with the rise of temperature, while SELH in a DMSO-treated group decreased significantly. FIG. 11C is a line graph depicting surface plasmon resonance (SPR) analysis of the interaction between ISP I Zenomycin with antioxidant components, including SELH, synthesized in bacteria. FIG. 11D presents western blots showing the decline, in a dose-dependent manner, of SELH levels in ISP 1-treated LN229 cells at 24 hours post-treatment. FIG. 11E presents western blots that show decreased SELH levels in ISP I-treated glioblastoma cell lines (T98G, U118, LN229, and U251) after treatment with 10 μg/mL of ISP I for 24 hours. FIG. 11F presents results from a cycloheximide pulse chase assay and immunoblotting, which evidence a decrease of SELH protein half-life in ISP I-treated LN229 cells. FIG. 11G is a line graph that quantifies immunoblot band results from FIG. 11F. FIG. 11H depicts results, obtained by knocking out SELH in LN229 cells (KO #2), showing a resistance to ISP I and, from western blots, no detectable SELH expression; calculated IC50 values also are tabulated. FIG. 11I presents data from a knock-down of SELH in LN229 cells, resulting in a decrease of cell proliferation. Western blots show no detectable expression of SELH in the LN229 cells two days after SELH siRNA transfection. As a control, a CCK8 assay was performed to measure cell proliferation for wild-type LN229 cells. FIG. 11J depict cell-cycle analysis data for SELH-deficient LN229 and U251 cells. FIG. 11K depicts flow cytometry data that evidence G0/G1 arrest in the SELH-deficient LN229 and U251 cells. FIG. 11L shows flow cytometry data from an apoptosis (Annexin-V stain) analysis of SELH-deficient LN229 and U251 cells with ISP I treatment. FIG. 11M presents in bar graph format results of apoptosis analysis (four independent wells) of the SELH-deficient LN229 and U251 cells. Expression of GAPDH served as an internal control in FIGS. 11D-F, H and I. All data are shown as mean±s.e.m. P value: *p<0.05; **p<0.01; ***p<0.001;

FIG. 12 is a combined graphic, schematic and tabular presentation of ISP I's targeting to SELH in RCC cells. FIG. 12A depicts wester blotting results for SELH expression in 786-O cells. ISP I-protected SELH was observed along with a rise in temperature, while SELH decreased significantly with DMSO treatment. FIG. 12B portrays western blots that show decreased SELH levels in 786-O and RCC4 cells after treatment with ISP I (10 μg/ml) for 24 hours. FIG. 12C provides an overview of results from SELH knock out in 786-O and RCC4 cells, resulting in resistance to ISP I. Western blots show low expression of SELH in the RCC cells. The tabulated values compare ISP I IC50 for wild-type and knock-out cells from each RCC line. FIG. 12D depicts data from a cell-cycle analysis of SELH-deficient 786-O and RCC4 cells. FIG. 12E provides in bar graph format an overview of cell-cycle progression data, showing clear G0/G1 arrest in SELH-deficient cells. FIG. 12F portrays results from an Annexin-V apoptosis analysis of SELH-deficient 786-O and RCC4 cells treated with ISP I. FIG. 12G summarizes in bar graph format results of the apoptosis analysis (four independent wells). Expression of GAPDH serves as an internal control in FIGS. 12B and 12C. All data are shown as mean±s.e.m. P value: *p<0.05; **p<0.01; ***p<0.001;

FIG. 13 is a combined schematic, graphic and photographic presentation of ISP I's suppression of tumor growth in glioblastoma xenograft mouse model and reduction of tumor burden in a melanoma lung metastasis mouse model. FIG. 13A is a schematic diagram of the progression of in vivo experimentation in this regard. NSG mice received intracranial injection of 1×10⁵ LN229-luc cells. Seven days after implantation, the resultant tumors were imaged and mice were randomized into two groups: untreated (N=8) and ISP I-treated (N=8). The mice were injected intraperitoneally with ISP I (66 mg/kg body weight) every day. FIG. 13B is a line graph of results from bioluminescence imaging that was used to follow tumor progression. The luminescence signal showed reduced LN229-luc tumor burden compared with the untreated group; p value was calculated by two-way ANOVA (****p<0.001). FIG. 13C presents tumor-derived bioluminescence images for three mice, showing a complete response induced by ISP I on day 24. FIG. 13D is a schematic diagram showing progression of the in vivo experiment. C57/B16 mice received tail vein injection of 2×10⁵ murine B16 cells on day 0. Mice were randomized into three groups: untreated (N=9), ISP I-treated (N=9) group, and Carrimycin-treated (N=9). Every day the mice were injected intraperitoneally with ISP I (35 mg/kg body weight) or were subjected to oral gavage with Carrimycin (56 mg/kg). After 12 days, the lungs were photographed and the melanoma spots on the lung were counted, as shown in FIGS. 13E and 13F, respectively. With reference to FIGS. 13G, 13H and 11I, the knock out of SELH in B16 cells resulted in a significant decrease of lung metastasis. Thus, FIG. 13G presents western blots showing no detectable expression of SELH in the B16 cells. FIG. 13H presents photographs taken of the lungs at 12 days after tail vein injection of B16 or SELH-deficient B16 cells (N=5 per group), and FIG. 13I depicts the results when melanoma spots on the lungs were counted. All data are shown as mean±s.e.m. P value: *p<0.05; **p<0.01; ***p<0.001;

FIG. 14 is a combined graphic, schematic, tabular and photomicrographic presentation of ISP I's suppressing tumor growth in RCC and meningioma xenograph mouse models. FIG. 14A is a schematic diagram of the in vivo experiment. NSG mice were injected subcutaneously in the flank with 786-O cells (1×10⁷). One week later the tumor-bearing mice were randomly assigned to two groups and treated daily with normal saline or ISP I (35 mg/kg), administered intraperitoneally. FIG. 14B is a line graph of tumor volume data, calculated based on caliper measurements. The tumor growth curve shows reduced 786-O tumor burden compared with the untreated group; p value was calculated by two-way ANOVA (***p<0.001). FIG. 14C shows, for 786-O xenograft, photographic images of tissue samples from control and ISP I groups, with corresponding tumor-weight data in bar graph format. Tumors were excised and weighed at the end of the experiment (18 days after treatment). FIG. 14D is a line graph of CCK8 results that delineate an ISP I dose-response curve for each of three meningioma cell lines, IOMM, JEN and CH-157, for 48 hours. FIG. 14E is a table listing IC50 values calculated for each of the aforementioned cell lines. FIG. 14F is a schematic diagram of the experiment in vivo. Nude mice were injected subcutaneously in the flank with IOMM cells (5×10⁶). One week later the tumor-bearing mice were assigned randomly to two groups and were treated daily with normal saline or ISP I (35 mg/kg), administered intraperitoneally. FIG. 14G is a line graph of data showing the effect of ISP I on tumor size. Tumors were measured with a caliper and volume was calculated. The tumor growth curve reflected a reduced IOMM tumor burden compared with the untreated group; p value was calculated by two-way ANOVA (***p<0.001). FIG. 14H provides a photographic comparison, for control group versus ISP I group, of IOMM xenograft tumor size. Tumors were excised and weighed at the end of the experiment (18 days after treatment). All data are shown as mean±s.e.m.

It should be noted that these drawings and written description are not intended to limit the scope of the present disclosure in any way, but rather to illustrate the concept of the present disclosure by reference to specific embodiments for those skilled in the art.

### DETAILED DESCRIPTION

To make the objectives, technical solutions, and advantages of embodiments of the present disclosure clearer, the technical solutions in the embodiments will be clearly and completely described below in combination with the accompanying drawings in the embodiments of the present disclosure. The following embodiments are used to describe the present disclosure but not to limit the scope of the present disclosure.

### Embodiment 1 Heterologous Expression of SelH

1. Vector Construction

A Cys-deficient Escherichia coli expression system was adopted, which is an effective method to prepare selenoprotein found in recent years. Itsbasic principle is as follows: the characteristic that Cys transfer RNA (tRNACys) can bind to Sec was utilized, and a culture medium which is lack of cysteine (Cys) and rich in Selenium cysteine (Sec) was used for culturing a strain, so that Sec was successfully incorporated into newly expressed protein. According to existing literature reports, a human SelH gene sequence was obtained. Selenocysteine of the SelH is in the middle of the sequence and was terminated here during prokaryotic expression, so that the selenocysteine was mutated from a triple codon TGA of the selenocysteine into TGC, which was synthesized by the Sangon Bioengineering (Shanghai) Co., Ltd. In addition, the adopted SPP system was a single protein production system, and a principle of the SPP system was to induce MazF enzyme (mRNA interfering enzyme for cutting RNA on an ACA nucleotide sequence) to cause growth arrest of thalli. The enzyme can specifically recognize and cut the ACA sequence and prevent synthesis of other background proteins in bacteria. Therefore, when mRNA of the desired target protein was encoded, if the mRNA can be designed into a form lacking an ACA base triplet according to an amino acid encoding rule and was induced at 15° C. by using a pCold vector in cells expressing MazF, the mRNA was not enzymatically hydrolyzed, only a protein derived from the mRNA was generated, and there was little synthesis of other cell proteins.

A pColdI-selH plasmid was transformed into BL21 (DE3) Cys-deficient Escherichia coli, so as to obtain a defective expression bacterium pColdI-selH. Sec was added during culture, making it become selenium-containing protein. The target protein was purified by using Ni²⁺ column chelating chromatography, and impure protein was eluted by using an imidazole eluent of different concentrations to finally obtain the target protein. Soluble SelH protein was finally obtained, and SDS-PAGE (sodium dodecyl sulfate polyacrylamide gel electrophoresis) analysis was carried out.

### Embodiment 2

(1) Screening of small molecule drugs by taking SelH as a target

A surface plasmon resonance (SPR) method was adopted to detect the interaction condition of a small molecule and a protein, so that the screening of drugs targeting SelH was carried out.

1. Protein Immobilization

Purified SelH was diluted into a 50 μg/mL protein solution by using 10 mM sodium acetate with a pH value of 5.5, the purified SelH was fixed onto a CM5 chip by an amino coupling method by using Biacore 8K (GE Healthcard, Sweden), and an RU value was recorded.

2. Sample Preparation

An isopentenyl or isovaleryl substituted coumarin compound, or a triterpenoid compound with an isopentenyl-like structure (the compound specifically includes aurapten, protopanaxadiol, iso-imperatorin, decursin, osthol, notoginsenoside R1 and shionon) was dissolved in 100% DMSO to be prepared into solutions containing 5% DMSO of different concentrations (0, 31.25, 62.5, 125, 250 and 500 μM) by using a 1.05×PBS-P⁺ buffer solution (GE Healthcare, obtained by diluting 10×PBS-P⁺).

3. Binding Experiment

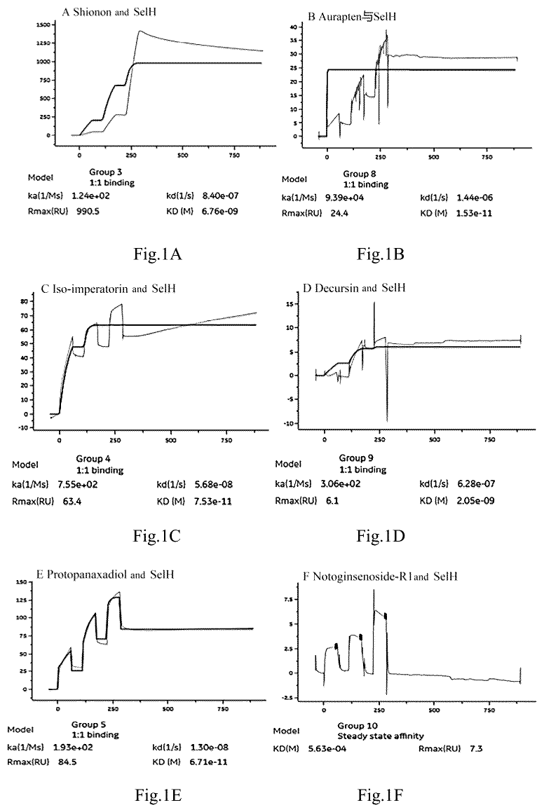

A single-cycle kinetics method was adopted, the 1.05×PBS-P⁺ buffer solution containing 5% DMSO was used as a Running Buffer, different compounds of different concentrations flowed through the SelH fixed onto the chip, wherein the binding time was 120 s, and the change condition of the RU value was recorded. An equilibrium dissociation constant (KD) was calculated by using software in Biacore 8K so as to evaluate the binding affinity between the protein and the compounds. The SPR detection result of the affinity between compounds and SelH in Embodiment 2 of the present disclosure is as shown in FIG. 1A-G, and the result statistics is as shown in Table 1.

The binding affinity was generally measured and reported by the equilibrium dissociation constant (KD) that is used to evaluate the intensity of the bimolecular interaction and to sort such intensities. The smaller a KD value, the greater the binding affinity of a ligand to its target.

(2) In-Vitro Anti-Tumor Activity Test of the Candidate Primarily Screened Drugs

1. Experimental Materials Human non-small cell lung cancer cells A549, mouse breast cancer cells 4T-1 and mouse melanoma B16-BL6 were purchased from American Type Current Collection (ATCC, Rockville, MD, USA). An RPMI-1640 culture solution containing 10% of fetal calf serum and 2% of glutamine was inoculated with the A549, 4T-1 and B16-BL6 cells, and the cells were incubated at 37° C. in a 5% CO₂ incubator.

1.2 Drugs and Reagents

The compounds in the step (1) were used as samples to be detected. The compounds were dissolved in DMSO under a sterile condition, and the obtained solutions were diluted to required concentrations by using an RPMI 1640 culture solution, wherein the final concentration of the DMSO was less than 0.5%.

The fetal calf serum is from Beijing Yuanheng Shengma Biological Technology Research Institute.

Trypsin, the glutamine, penicillin, streptomycin, dimethyl sulfoxide (DMSO) and methyl thiazolyl tetrazolium (MTT) were purchased from the American Sigma company.

1.3 Instruments

Carbon dioxide incubator (NuAir, USA), Enzyme-linked immunosorbent assay analyzer (Tecan, Austria), 96-well culture plate (Corning, USA) and Inverted microscope (Motic, China).

2. Experimental Method

MTT reduction method is used.

2.1 Basic Principle:

Methyl thiazolyl tetrazolium [3-(4,5-dimethylibiazol-2-yl)-2,5-diphenyl-tetrazolium bromide, MTT] is a dye capable of accepting hydrogen atoms. The MTT can act on a respiratory chain in mitochondria of living cells, a tetrazolium ring is cracked under the action of succinate dehydrogenase and cytochrome c to generate blue purple formazan crystals. The generation amount of the formazan crystals is only in direct proportion to the number of the living cells (succinate dehydrogenase in dead cells disappears, and MTT cannot be reduced). After the formazan is dissolved by DMSO, an optical density value is measured by a microplate reader under a certain wavelength, and the survival rate of cells can be quantitatively measured.

2.2. Operation Steps:

A549, 4T-1 and B16-BL6 were used as adherent cells, adherent tumor cells in a logarithmic phase were selected, digested with pancreatin, and then prepared into a cell suspension of 5×10⁴/ml with a culture medium containing 10% of calf serum, a 96-well culture plate was inoculated with the cell suspension, wherein each well contained 100 μl of the cell suspension, and incubation was carried out for 24 h at 37° C. and 5% CO₂. Replacing was carried out with a new culture solution containing the samples to be detected of different concentrations in the experimental groups, replacing was carried out with a culture solution containing a same volume of solvent in the control group, three parallel wells were set in each group, and incubation was carried out for 48 hours at 37° C. and 5% CO₂. The supernate was discarded, careful washing was carried out with PBS twice, 100 μl of a freshly prepared culture medium containing 0.5 mg/ml of MTT was added into each well, and incubation was continued to be carried out at 37° C. for 4 h. The supernatant was carefully discarded, 150 μl of DMSO was added, uniform mixing was carried out for 10 min by using a micro oscillator, and the optical density value was measured at 492 nm by using a microplate reader.

2.3. Result Evaluation:

The inhibition ratio of the drugs on the growth of tumor cells is calculated according to the following formula:

Tumor cell growth inhibition ratio (%)=[A₄₉₂ (negative control)−A₄₉₂ (dosing group)]/A₄₉₂ (negative control)×100%

The median inhibitory concentration (IC₅₀) of the sample is calculated.

Wherein IC₅₀ is a median inhibitory concentration, a drug concentration required for inhibiting cell growth, virus replication and the like by 50%.

The result of the inhibition effect of the compounds on the growth of in-vitro tumor cells is shown in Table 1.

TABLE 1
Dissociation constant KD of compounds and SelH and
its cytotoxic effect on 4T1, B16-BL6 and A549

| | | IC₅₀ (μM) | | |
|---|---|---|---|---|
| Compund | KD (M) | 4T1 | B16-BL6 | A549 |
| Aurapten | 1.53E−11 | 37.67 | 61.86 | 51.85 |
| Protopanaxadiol | 6.71E−11 | 30.85 | 57.99 | 54.43 |
| Iso-imperatorin | 7.53E−11 | 48.94 | 75.15 | 72.5 |
| Decursin | 2.05E−09 | 38.20 | 67.79 | 60.03 |
| Osthol | 8.51E−08 | 67.42 | 100.1 | 81.16 |
| Notoginsenoside R1 | 5.63E−04 | 100.5 | 110.8 | 126.3 |
| Shionon | 6.76E−09 | 82.4 | 93.90 | 92.7 |

The affinity between the compounds and selenoprotein H is tested by adopting the surface plasmon resonance (Biacore) technology. It can be seen from Table 1 that in the above compounds, the binding activity of protopanaxadiol and iso-imperatorin to the SelH is the strongest, and the binding activity of notoginsenoside R1 to the SelH is the weakest.

The MTT method shows that the isopentenyl or isovaleryl substituted coumarin or the triterpenoid compound with the isopentenyl-like structure shows an obvious inhibition effect on the proliferation of the mouse breast cancer 4T1 cells, mouse melanoma B16-BL6 cells and human lung cancer A549 cells.

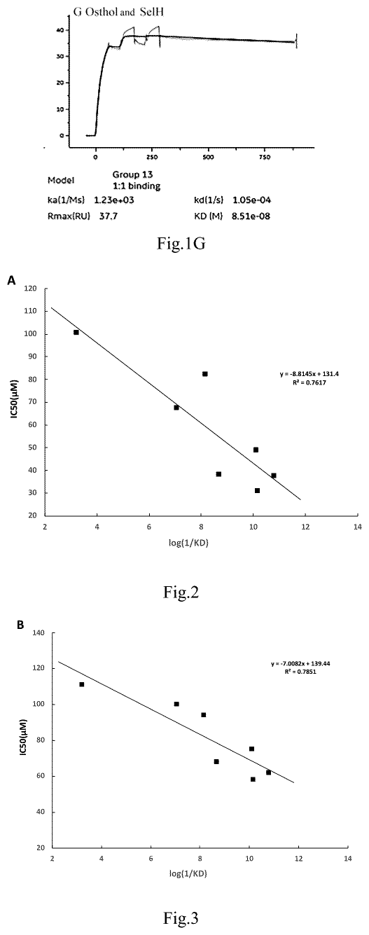

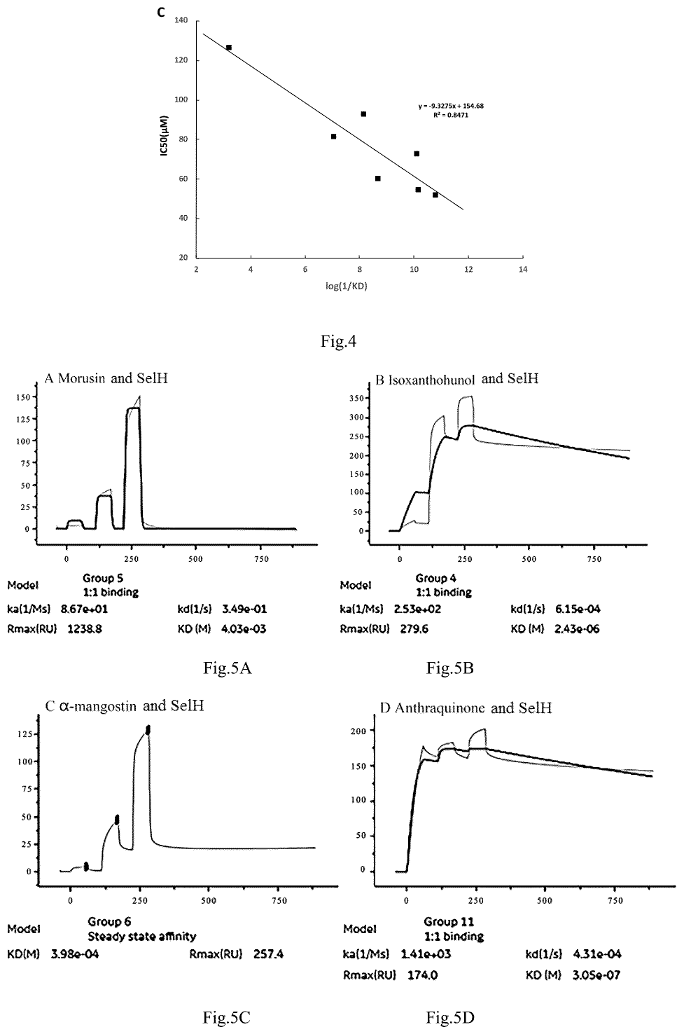

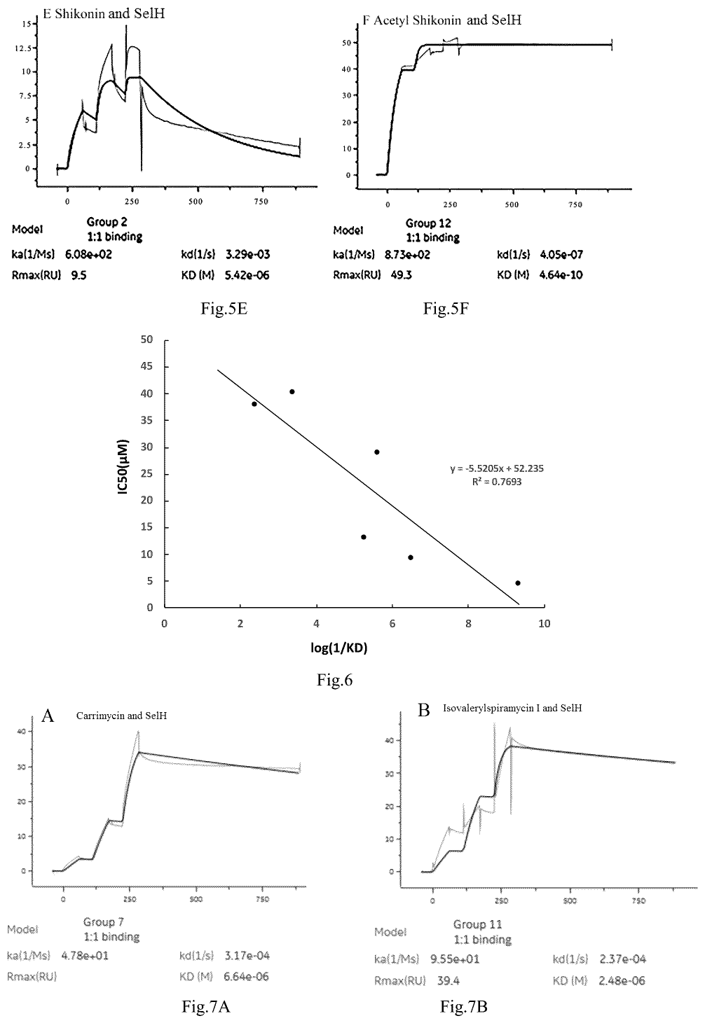

As shown in FIGS. 2-4, the correlation analysis shows that the affinity between the compounds and the selenoprotein H, and the inhibition effect on tumor cells have obvious correlation, and the inhibition effect on the tumor cells is enhanced along with the increase of the affinity. Therefore, for the compounds, the effect of the compounds on inhibiting proliferation of breast cancer cells, melanoma cells and lung cancer cells can be inferred by measuring the affinity between the compounds and the selenoprotein H. And the selenoprotein H is suitable for being used as effective targets for high-throughput screening of anti-tumor drugs.

### Embodiment 3

(1) Screening of small molecule drugs by taking SelH as a target A surface plasmon resonance (SPR) method was adopted to detect the interaction condition of a small molecule and a protein, so that the screening of drugs targeting SelH was carried out.

1. Protein Immobilization

Purified SelH was diluted into a 50 μg/mL protein solution by using 10 mM sodium acetate with a pH value of 5.5, the purified SelH was fixed onto a CM5 chip by an amino coupling method by using Biacore 8K (GE Healthcard, Sweden), and an RU value was recorded.

2. Sample Preparation

An isopentenyl or isovaleryl substituted flavonoids or shikonin compounds (the compound specifically includes Acetyl Shikonin, Anthraquinone, Isoxanthohunol, α-mangostin, Morusin, Shikonin) was dissolved in 100% DMSO to be prepared into solutions containing 5% DMSO of different concentrations (0, 31.25, 62.5, 125, 250 and 500 μM) by using a 1.05×PBS-P⁺ buffer solution (GE Healthcare, obtained by diluting 10×PBS-P⁺).

3. Binding Experiment

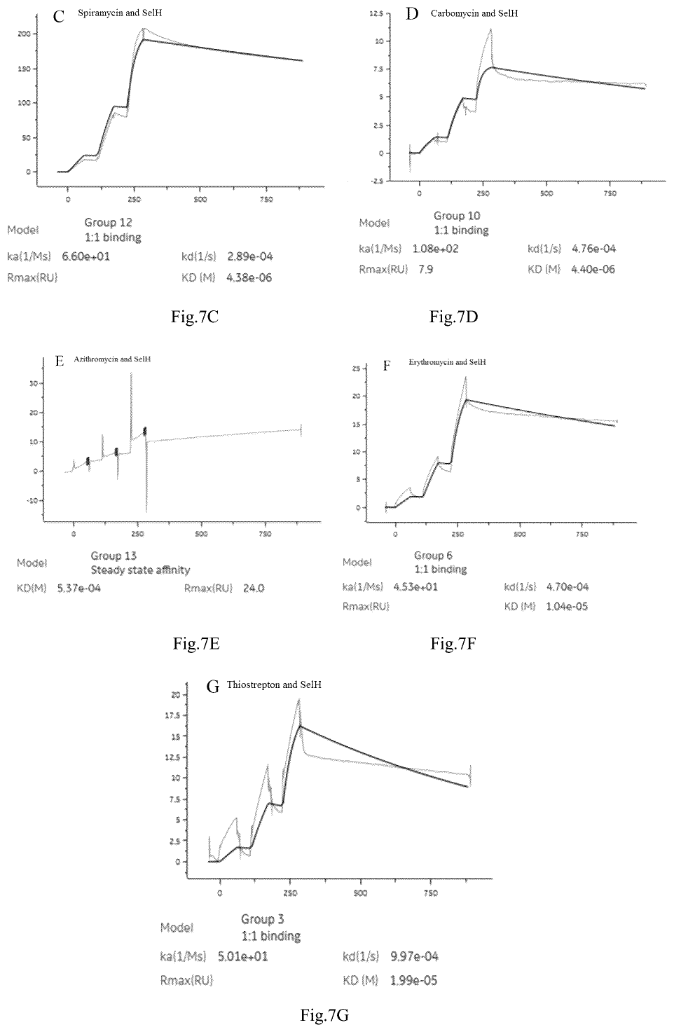

A single-cycle kinetics method was adopted, the 1.05×PBS-P⁺ buffer solution containing 5% DMSO was used as a Running Buffer, different small molecule compounds of different concentrations flowed through the SelH fixed onto the chip, wherein the binding time was 120 s, and the change condition of the RU value was recorded. An equilibrium dissociation constant (KD) was calculated by using software in Biacore 8K so as to evaluate the binding affinity between the protein and the small molecule compounds. The SPR detection result of the affinity between compounds and SelH in Embodiment 3 of the present disclosure is as shown in FIG. 5A-F, and the result statistics is as shown in Table 2.

The binding affinity was generally measured and reported by the equilibrium dissociation constant (KD) that is used to evaluate the intensity of the bimolecular interaction and to sort such intensities. The smaller a KD value, the greater the binding affinity of a ligand to its target.

(2) In-Vitro Anti-Tumor Activity Test of the Candidate Primarily Screened Drugs

1. Experimental Materials

The mouse melanoma cell B16-BL6 was purchased from American Type Current Collection (ATCC, Rockville, MD, USA). An RPMI-1640 culture solution containing 10% of fetal calf serum and 2% of glutamine was inoculated with the B16-BL6 cells, and the cells were incubated at 37° C. in a 5% CO₂ incubator.

1.2 Drugs and Reagents

The compounds in the step (1) were used as samples to be detected. The compounds were dissolved in DMSO under a sterile condition, and the obtained solutions were diluted to required concentrations by using an RPMI 1640 culture solution, wherein the final concentration of the DMSO was less than 0.5%.

The fetal calf serum is from Beijing Yuanheng Shengma Biological Technology Research Institute.

Trypsin, the glutamine, penicillin, streptomycin, dimethyl sulfoxide (DMSO) and methyl thiazolyl tetrazolium (MTT) were purchased from the American Sigma company.

1.3 Instruments

Carbon dioxide incubator (NuAir, USA), Enzyme-linked immunosorbent assay analyzer (Tecan, Austria), 96-well culture plate (Corning, USA) and Inverted microscope (Motic, China).

2. Experimental Method

MTT reduction method is used.

2.1 Basic Principle:

Methyl thiazolyl tetrazolium [3-(4,5-dimethylibiazol-2-yl)-2,5-diphenyl-tetrazolium bromide, MTT] is a dye capable of accepting hydrogen atoms. The MTT can act on a respiratory chain in mitochondria of living cells, a tetrazolium ring is cracked under the action of succinate dehydrogenase and cytochrome c to generate blue purple formazan crystals. The generation amount of the formazan crystals is only in direct proportion to the number of the living cells (succinate dehydrogenase in dead cells disappears, and MTT cannot be reduced). After the formazan is dissolved by DMSO, an optical density value is measured by a microplate reader under a certain wavelength, and the survival rate of cells can be quantitatively measured.

2.2. Operation Steps:

The B16-BL6 cells were used as adherent cells, adherent tumor cells in a logarithmic phase were selected, digested with pancreatin, and then prepared into a cell suspension of 5×10⁴/ml with a culture medium containing 10% of calf serum, a 96-well culture plate was inoculated with the cell suspension, wherein each well contained 100 μl of the cell suspension, and incubation was carried out for 24 h at 37° C. and 5% CO₂. Replacing was carried out with a new culture solution containing the samples to be detected (compound in table 2) of different concentrations in the experimental groups, replacing was carried out with a culture solution containing a same volume of solvent in the control group. Three parallel wells were set in each group, and incubation was carried out for 48 hours at 37° C. and 5% CO₂. The supernate was discarded, careful washing was carried out with PBS twice, 100 μl of a freshly prepared culture medium containing 0.5 mg/ml of MTT was added into each well, and incubation was continued to be carried out at 37° C. for 4 h. The supernatant was carefully discarded, 150 μl of DMSO was added, uniform mixing was carried out for 10 min by using a micro oscillator, and the optical density value was measured at 492 nm by using a microplate reader.

2.3. Result Evaluation:

The inhibition ratio of the drugs on the growth of tumor cells is calculated according to the following formula:

Tumor cell growth inhibition ratio (%)=[A₄₉₂ (negative control)−A₄₉₂ (dosing group)]/A₄₉₂ (negative control)×100%

The median inhibitory concentration (IC₅₀) of the sample is calculated.

Wherein IC₅₀ is a median inhibitory concentration, a drug concentration required for inhibiting cell growth, virus replication and the like by 50%.

The result of the inhibition effect of the compounds on the growth of in-vitro tumor cells is shown in Table 2.

TABLE 2
Dissociation constant KD of compounds and
SelH and its cytotoxic effect on B16-BL6

| Compound | KD(M) | IC₅₀(μM) |
|---|---|---|
| Acetyl Shikonin | 4.64E−10 | 4.44 |
| Anthraquinone | 3.05E−07 | 9.34 |
| Isoxanthohunol | 2.43E−06 | 29.03 |
| α-mangostin | 3.98E−04 | 40.19 |
| Morusin | 4.03E−03 | 37.86 |
| Shikonin | 5.42E−06 | 13.03 |

The affinity between the compounds and selenoprotein H is tested by adopting the surface plasmon resonance (Biacore) technology. It can be seen from Table 2 that in the above compounds, the binding activity of Acetyl Shikonin to the SelH is the strongest, and the binding activity of Morusin to the SelH is the weakest.

The MTT method shows that the isopentenyl substituted flavonoids or shikonin compounds with high affinity significantly inhibited the proliferation of mouse melanoma B16-BL6 cells.

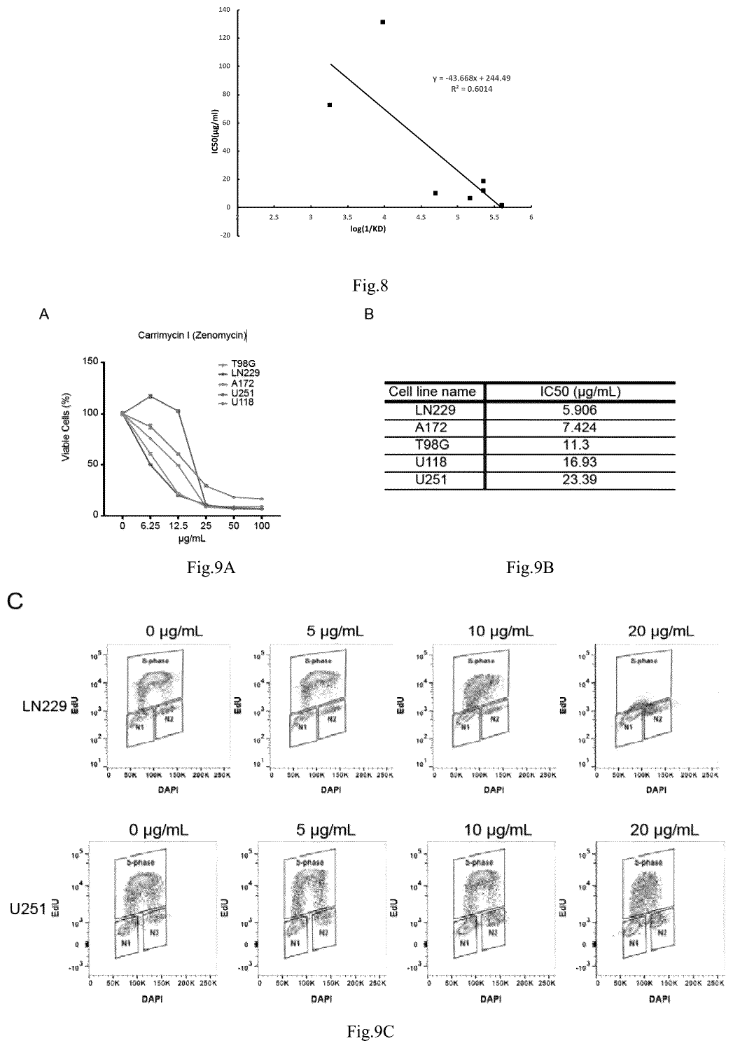

As shown in FIGS. 6, the correlation analysis shows that the affinity between the compounds and the selenoprotein H, and the inhibition effect on B16-BL6 cells have obvious correlation, and the inhibition effect on the B16-BL6 cells is enhanced along with the increase of the affinity. Therefore, for the compounds, the effect of the compounds on inhibiting proliferation of mouse melanoma B16-BL6 cells can be inferred by measuring the affinity between the compounds and the selenoprotein H. And the selenoprotein H is suitable for being used as effective targets for high-throughput screening of anti-tumor drugs.

### Embodiment 4

(1) Screening of Macrolide and Cyclic Peptide Drugs by Taking SelH as a Target

A surface plasmon resonance (SPR) method was adopted to detect the interaction condition of a small molecule and a protein, so that the screening of drugs targeting SelH was carried out.

1. Protein Immobilization

Purified SelH was diluted into a 50 μg/mL protein solution by using 10 mM sodium acetate with a pH value of 5.5, the purified SelH was fixed onto a CM5 chip by an amino coupling method by using Biacore 8K (GE Healthcard, Sweden), and an RU value was recorded.

2. Sample Preparation

The macrolide compound or cyclic peptide compound (the compounds specifically include carrimycin, isovalerylspiramycin I, spiramycin, carbomycin, azithromycin, erythromycin and thiostrepton) was dissolved in 100% DMSO to be prepared into solutions containing 5% DMSO of different concentrations (0, 31.25, 62.5, 125, 250 and 500 μM) by using a 1.05×PBS-P⁺ buffer solution (GE Healthcare, obtained by diluting 10×PBS-P⁺).

3. Binding Experiment

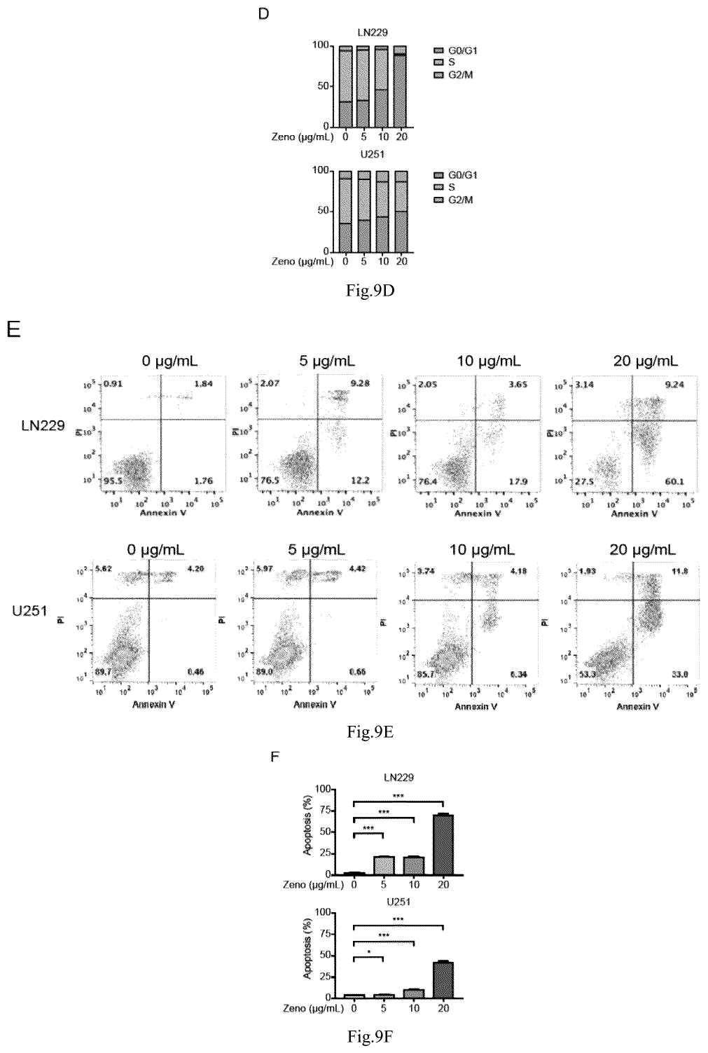

A single-cycle kinetics method was adopted, the 1.05×PBS-P⁺ buffer solution containing 5% DMSO was used as a Running Buffer, different compounds of different concentrations flowed through the SelH fixed onto the chip, wherein the binding time was 120 s, and the change condition of the RU value was recorded. An equilibrium dissociation constant (KD) was calculated by using software in Biacore 8K so as to evaluate the binding affinity between the protein and the compounds. The SPR detection result of the affinity between compounds and SelH in Embodiment 4 of the present disclosure is as shown in FIG. 7A-G, and the result statistics is as shown in Table 3. The binding affinity was generally measured and reported by the equilibrium dissociation constant (KD) that is used to evaluate the intensity of the bimolecular interaction and to sort such intensities. The smaller a KD value, the greater the binding affinity of a ligand to its target.

(2) In-Vitro Anti-Tumor Activity Test of the Candidate Primarily Screened Drugs

1. Experimental Materials

Human non-small cell lung cancer cells A549 was purchased from American Type Current Collection (ATCC, Rockville, MD, USA). An RPMI-1640 culture solution containing 10% of fetal calf serum and 2% of glutamine was inoculated with the A549 cells, and the cells were incubated at 37° C. in a 5% CO₂ incubator.

1.2 Drugs and Reagents

The compounds in the step (1) were used as samples to be detected. The compounds were dissolved in DMSO under a sterile condition, and the obtained solutions were diluted to required concentrations by using an RPMI 1640 culture solution, wherein the final concentration of the DMSO was less than 0.5%.

The fetal calf serum is from Beijing Yuanheng Shengma Biological Technology Research Institute.

Trypsin, the glutamine, penicillin, streptomycin, dimethyl sulfoxide (DMSO) and methyl thiazolyl tetrazolium (MTT) were purchased from the American Sigma company.

1.3 Instruments

Carbon dioxide incubator (NuAir, USA), Enzyme-linked immunosorbent assay analyzer (Tecan, Austria), 96-well culture plate (Corning, USA) and Inverted microscope (Motic, China).

2. Experimental Method

MTT reduction method is used.

2.1 Basic Principle:

Methyl thiazolyl tetrazolium [3-(4,5-dimethylibiazol-2-yl)-2,5-diphenyl-tetrazolium bromide, MTT] is a dye capable of accepting hydrogen atoms. The MTT can act on a respiratory chain in mitochondria of living cells, a tetrazolium ring is cracked under the action of succinate dehydrogenase and cytochrome c to generate blue purple formazan crystals. The generation amount of the formazan crystals is only in direct proportion to the number of the living cells (succinate dehydrogenase in dead cells disappears, and MTT cannot be reduced). After the formazan is dissolved by DMSO, an optical density value is measured by a microplate reader under a certain wavelength, and the survival rate of cells can be quantitatively measured.

2.2. Operation Steps:

A549 cells were used as adherent cells, adherent tumor cells in a logarithmic phase were selected, digested with pancreatin, and then prepared into a cell suspension of 5×10⁴/ml with a culture medium containing 10% of calf serum. The 96-well culture plate was inoculated with the cell suspension, wherein each well contained 100 μl of the cell suspension, and incubation was carried out for 24 h at 37° C. and 5% CO₂. Replacing was carried out with a new culture solution containing the samples to be detected (compound in table 3) of different concentrations in the experimental groups, replacing was carried out with a culture solution containing a same volume of solvent in the control group. Three parallel wells were set in each group, and incubation was carried out for 48 hours at 37° C. and 5% CO₂. The supernate was discarded, careful washing was carried out with PBS twice, 100 μl of a freshly prepared culture medium containing 0.5 mg/ml of MTT was added into each well, and incubation was continued to be carried out at 37° C. for 4 h. The supernatant was carefully discarded, 150 μl of DMSO was added, uniform mixing was carried out for 10 min by using a micro oscillator, and the optical density value was measured at 492 nm by using a microplate reader.

2.3. Result Evaluation:

The inhibition ratio of the drugs on the growth of tumor cells is calculated according to the following formula:

Tumor cell growth inhibition ratio (%)=[A₄₉₂(negative control)−A₄₉₂(dosing group)]/A₄₉₂ (negative control)×100%

The median inhibitory concentration (IC₅₀) of the sample is calculated.

Wherein IC₅₀ is a median inhibitory concentration, a drug concentration required for inhibiting cell growth, virus replication and the like by 50%.

The result of the inhibition effect of the compounds on the growth of in-vitro tumor cells is shown in Table 3.

TABLE 3
Binding of macrolides and cyclic peptides
to SelH and cytotoxicity to A549

| | | A549 |
|---|---|---|
| Compound | KD(M) | IC₅₀ (μg/ml) |
| Carrimycin | 6.64e−06 | 6.11 |
| Isovalerylspiramycin I | 2.48e−06 | 1.29 |
| Spiramycin | 4.38e−06 | 18.33 |
| Carbomycin | 4.40e−06 | 11.79 |
| Azithromycin | 5.37e−04 | 72.43 |
| Erythromycin | 1.04e−05 | 230 |
| Thiostrepton | 1.99e−05 | 9.66 |

The affinity between the compounds and selenoprotein H is tested by adopting the surface plasmon resonance (Biacore) technology. It can be seen from Table 3 that in the above compounds, the binding activity of Isovalerylspiramycin I to the SelH is the strongest, and the binding activity of Azithromycin to the SelH is the weakest. The MTT method shows that the Isovalerylspiramycin I with high affinity significantly inhibited the human lung cancer A549 cells.

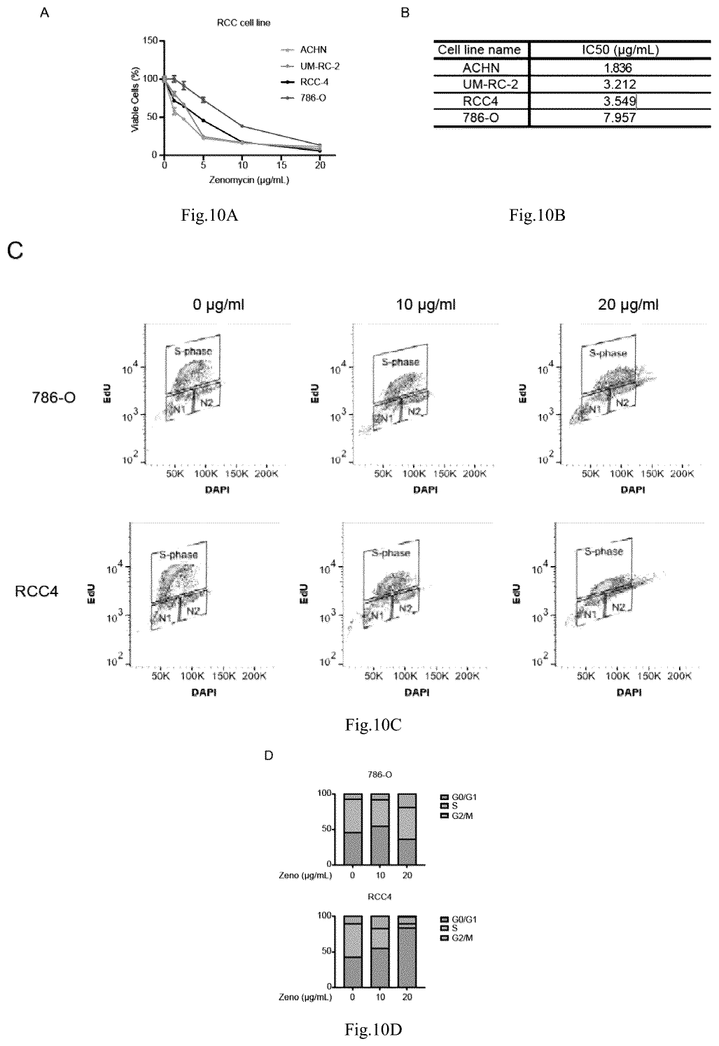

As shown in FIGS. 8, the correlation analysis shows that there is a significant correlation between the affinity of macrolides and cyclic peptides with selenoprotein h and their inhibitory effect on A549 cells, and the inhibition effect on the tumor cells is enhanced along with the increase of the affinity. Therefore, for the compounds, the effect of the compounds on inhibiting proliferation of lung cancer cells A549 can be inferred by measuring the affinity between the compounds and the selenoprotein H. And the selenoprotein H is suitable for being used as effective targets for high-throughput screening of anti-tumor drugs.

### Test Example 1

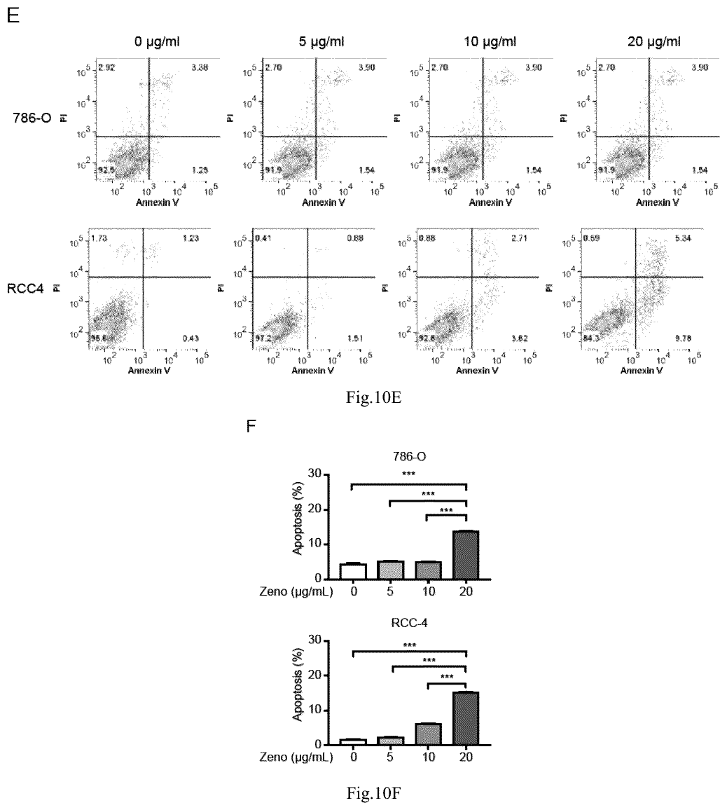

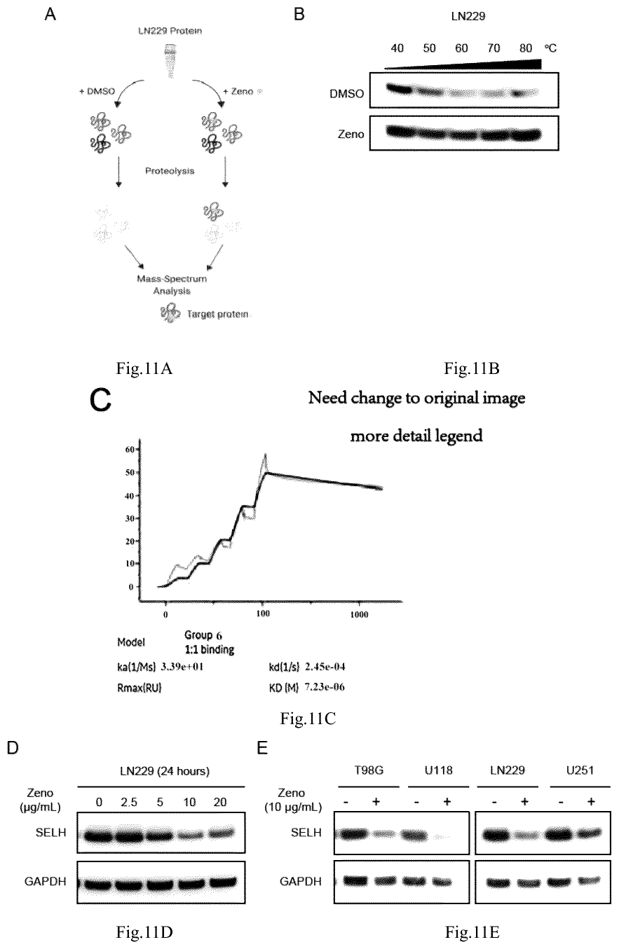

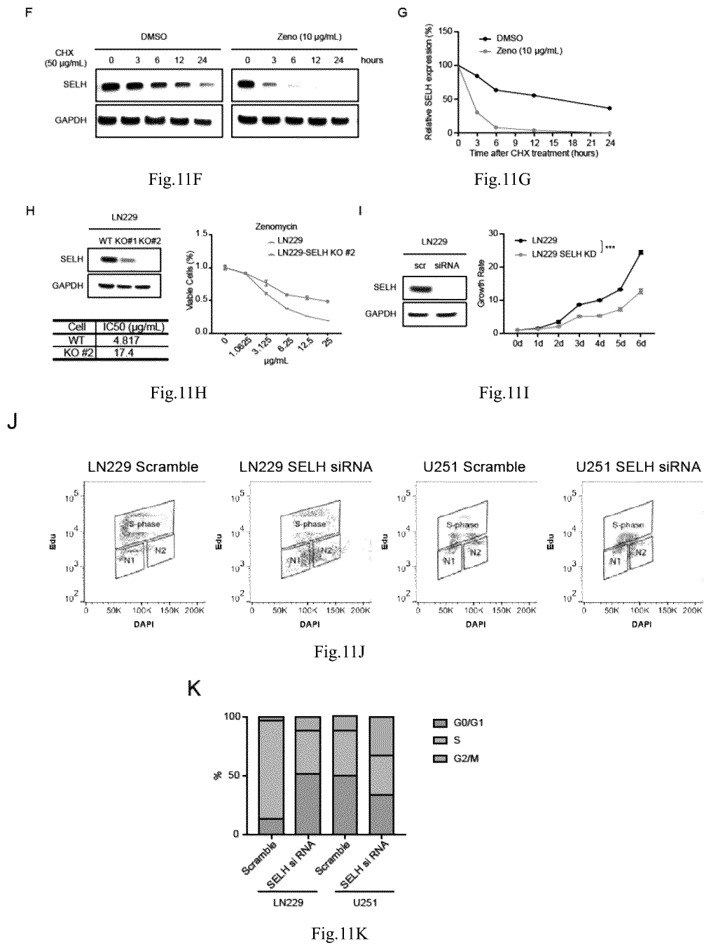

(1) To assess the cytotoxicity of ISPs I, II and III, five glioblastoma cell lines, T98G, U118, A172, LN229 and U251, were treated with serial doses of each ISP, respectively, for 48 hours. Cell viability of these cell lines were evaluated by CCK8 assay, and the 50% inhibitory concentration (IC50) was calculated (FIG. 9A, B). All glioblastoma cell lines were more sensitive to ISP I when compared to ISPs II and III. Out of the glioblastoma cell lines tested, LN229 appeared to be the most sensitive to Carrymycin's cytotoxic effect, while U251 appeared the least sensitive. Cell distribution in each phase of the cell cycle was assessed in LN229 and U251 cells by flow cytometry, followed by EdU and DAPI staining. The cell cycle analysis showed that ISP I resulted in a dose-dependent increase in the G0/G1 phase and a decrease in the S phase compared with the control cells (FIG. 9C, D). This finding indicates that treatment with ISP I induced a cell cycle arrest at the G0/G1 phase in treated cells. Further flow cytometric assessment with Annexin V stain, a marker for cell apoptosis, revealed that ISP I induced a dose-dependent apoptosis in treated cells (FIG. 9E, F).

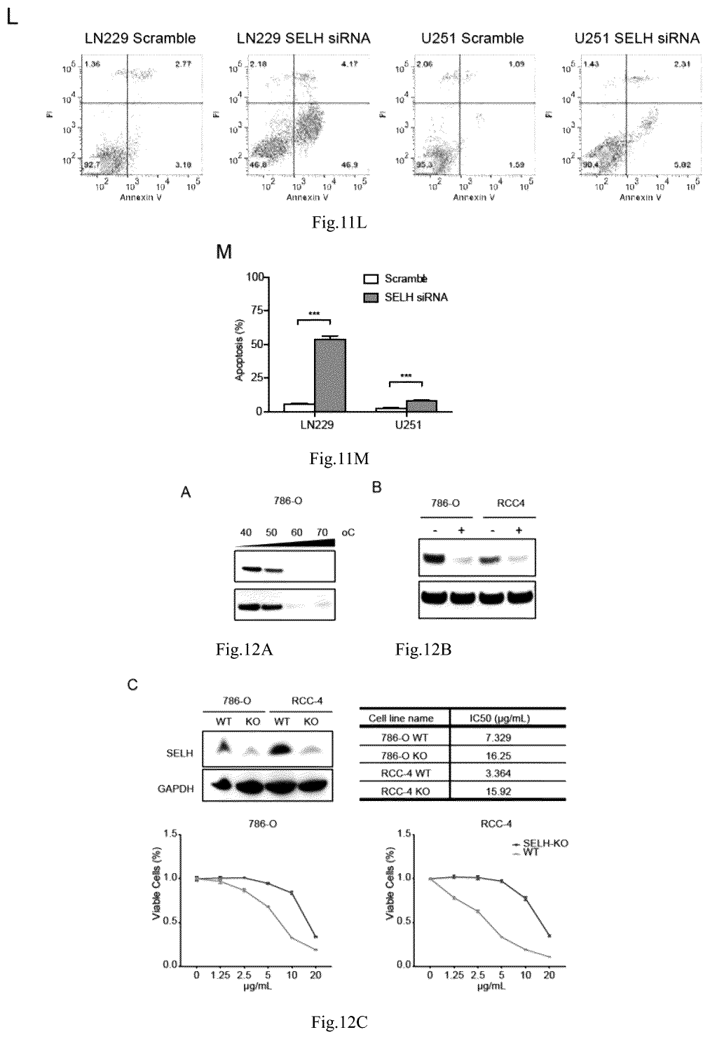

To confirm the cytotoxic effect observed in the glioblastoma cell lines, the inventors assessed ISP I's effect on renal cell carcinoma (RCC) cell lines (ACHN, UM-RC-2, RCC4 and 786-0). Cell viability was assessed similarly via CKK8 analysis. Out of the RCC cell lines tested, ACHN appeared to be the most sensitive to ISP I's cytotoxic effect while 786-O demonstrated to be the least sensitive (FIG. 10A, B). Consistent with glioblastoma cell line findings, flow cytometric analysis revealed that ISP I induced cell-cycle arrest in the G0/G1 phase in treated cells and likewise induced a dose-dependent apoptosis in treated cells. (FIG. 10C, D).

Taken together, these results indicate that ISP I inhibits cell proliferation by arresting cancer cells in the G0/G1 phase and inducing tumor cell apoptosis.

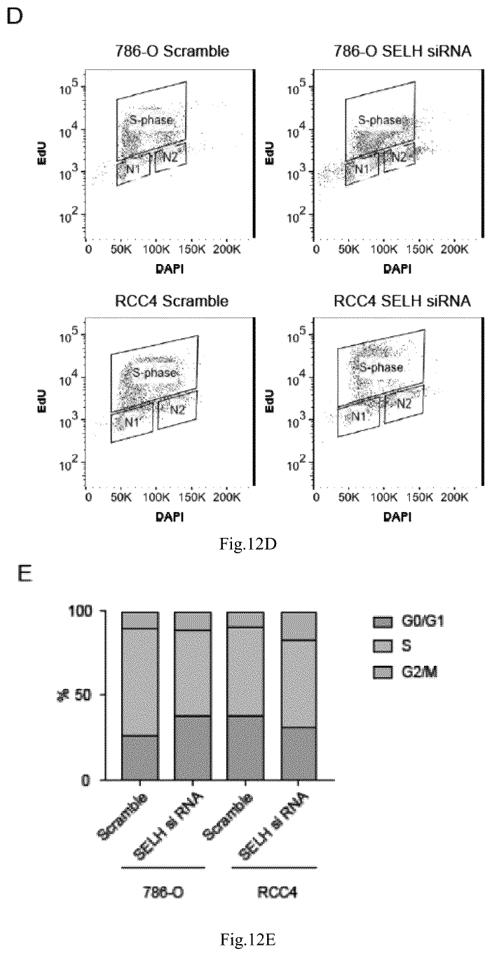

(2) To identify ISP I's molecular target, the inventors performed drug affinity responsive target stability (DARTS) assay in LN229 cells. The basic strategy of DARTS is shown in FIG. 11A.

The inventors discovered that the binding of ISP I to its target proteins temporarily locks them into a stable conformational structure, which prevents their recognition by proteases. After evading protease degradation, the identity of ISP I's target proteins was determined using mass spectroscopy. DARTS analysis results revealed that SELH was the most abundant primary protein present in ISP I-treated LN229 cells.

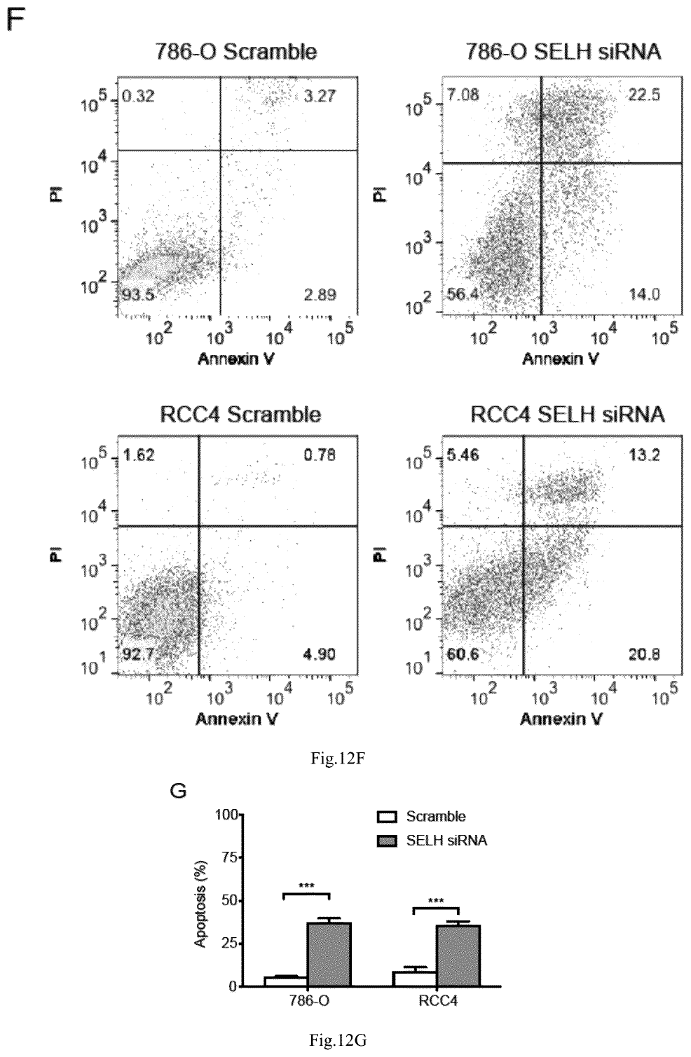

Next, the inventors used a thermo-stability assay to confirm SELH was targeted by ISP I in LN229 and 786-O cell lines. The principle of the assay is based on altered protein thermal stabilization/destabilization due to ligand binding in living cells. Indeed, western blot results demonstrated that the protective effect of ISP I on SELH was present over a range of increasing temperatures, an effect that was significantly decreased in the SELH from DMSO-treated groups. (FIG. 11B and FIG. 12A). To verify further the specificity of ISP I's targeting of SELH, the inventors designed a surface plasmon resonance assay to assess the interaction of ISP I with SELH synthesized in bacteria.

These results show that ISP I tightly binds to SELH but does not bind with the other proteins (FIG. 11C). Accordingly, the results indicate that the molecular target of ISP I is SELH.

To explore ISP I's effect on SELH, the inventors assessed the quantity of SELH protein in LN229 cells treated with different concentrations of ISP I. Treatment with ISP I reduced SELH protein expression in LN229 cells in a dose-dependent manner (FIG. 11D). ISP I's inhibitory effect on SELH expression also was confirmed in four glioblastoma cell lines, T98G, U118, LN229 and U251, and in two RCC cell lines, 786-O and RCC4 (FIG. 11E and FIG. 12B). A cycloheximide (CHX) chase assay was completed to assess ISP I's effect on SELH protein degradation. The CHX chase assay results confirm that treatment with ISP I reduced the SELH protein half-life, evidencing that ISP I promotes SELH protein degradation (FIG. 11F, G).

To confirm further that ISP I inhibits cell growth via a SELH-dependent mechanism, the inventors generated SELH-deficient LN229 cells and RCC cells (786-O and RCC4) with CRISPR/CAS9 and then treated them with ISP I. CCK8 assay results demonstrated that SELH-deficient cells were resistant to ISP I treatment when compared to wild-type LN229 cells (FIG. 11H and FIG. 12C). Next, the inventors knocked down SELH expression using siRNA in two glioblastoma cell lines (LN229 and U251) and two RCC cell lines (786-O and RCC4) to assess ISP I's effect on cell growth, proliferation and apoptosis. siRNA mediated knockdown of SELH resulted in a significantly decreased growth rate of LN229 cells (FIG. 11I) and significantly inhibited cell proliferation and apoptosis in the glioblastoma (LN229 and U251) and RCC cell lines (786-O and RCC4) (FIG. 11J-M and FIG. 12D-G). Together these data demonstrate that ISP I inhibited glioblastoma and RCC cell growth through the suppression of SELH expression.

(3) ISP I Suppresses Tumorigenesis and Metastasis In Vivo

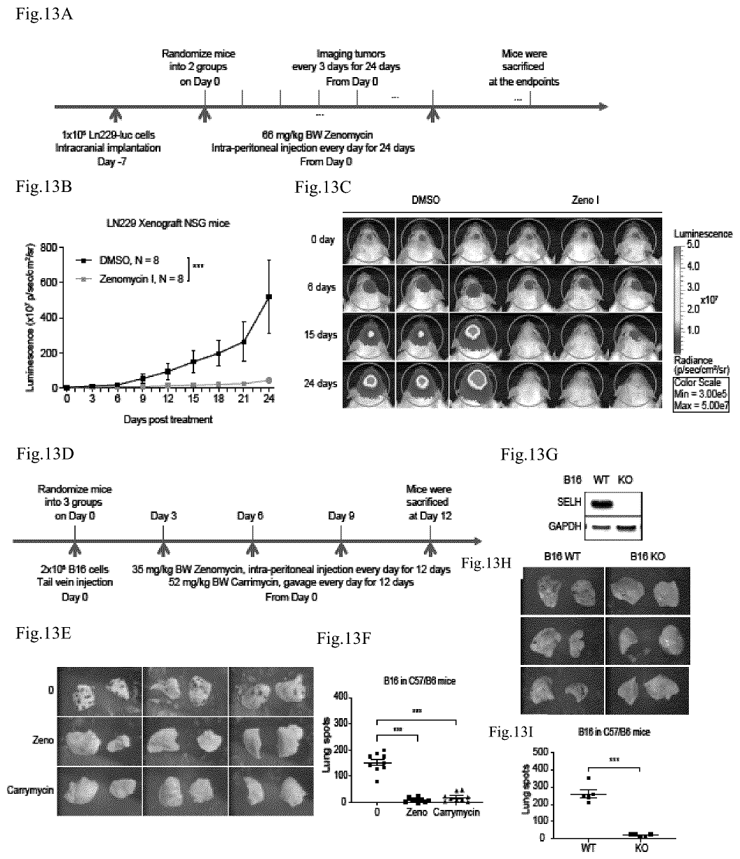

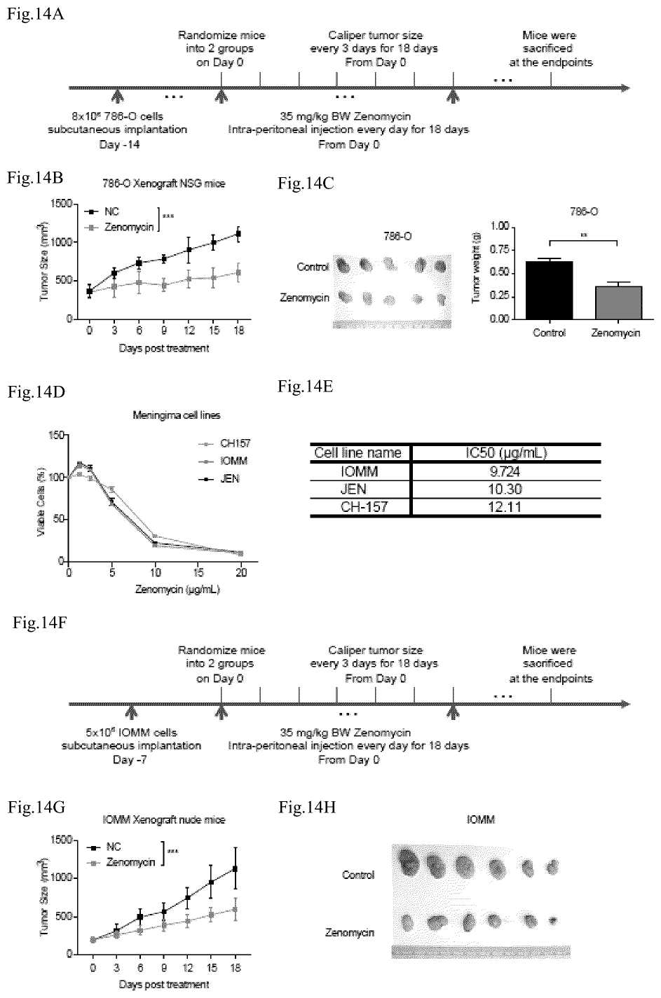

To assess whether ISP I could suppress tumor growth in vivo, the inventors investigated the tumor-suppressing effect of ISP I in three xenograft mouse models (FIG. 13A and FIG. 14A, F).

The inventors first assessed ISP I's anti-tumor activity in an intracranial mouse model (FIG. 13A). NSG mice were inoculated with 1×10⁵ LN229-luc cells in the right-frontal cortex. After 7 days, intracranial tumor growth was confirmed via noninvasive in vivo bioluminescence imaging and mice were randomized into ISP I or DMSO (control) treatment groups. Bioluminescence imaging results demonstrated that mice treated with ISP I demonstrated significantly reduced tumor growth when compared to mice in the DMSO treatment group (FIG. 13B, C).

Since ISP I demonstrated a cytotoxic effect on RCC (FIG. 10) and meningioma cell lines (IOMM, JEN, CH-157) (FIG. 14D, E), the inventors also assessed whether ISP I reduced tumor growth in 786-O (FIG. 14A-C) and IOMM (FIG. 14F-H) xenograft models. In both models, ISP I-treated mice demonstrated significantly reduced tumor size and weight compared to DMSO-treated groups (FIG. 14C, H). A dedicated veterinary histopathology review of major organs and standard clinical chemistry undertaken 24 days after treatment revealed no hematologic, renal, pancreatic or liver toxicities.

In addition to the aforementioned xenograft tumor models, the anti-tumor activity of ISP I was assessed in a metastatic murine melanoma (B16) model. Mice were intravenously injected with 2×10⁵ B16 cells and randomized into the following three treatment groups: ISP I (35 mg/kg), Carrimycin (56 mg/kg), Saline (control) (FIG. 13D). After 12 days of treatment, mice in the ISP I and Carrimycin treatment groups demonstrated significantly reduced lung tumor nodules when compared to saline treated mice (FIG. 13E, F). These data evidence that both ISP I and Carrimycin inhibit the formation of metastatic melanoma tumors.

To evaluate SELH's role in metastatic melanoma tumor formation, the inventors inoculated C57/B6 mice with SELH-deficient B16 cells or B16-wild-t e cells (FIG. 13G). Twelve days after tumor inoculation, mice injected with SELH-deficiency B16 cell demonstrated significantly re100002 26
2010.2

duced lung tumor nodules when compared to mice injected with B16-wild-type cells (FIG. 13H, I).

Taken together, these in vivo data indicate that ISP I induces a potent anti-tumor effect through the inhibition of SELH activity.

Methods

Cell Culture and Reagents

Glioblastoma cell lines (LN229, U118, T98G and A172) were derived from American Type Culture Collection (ATCC) of Manassas, VA. U251 was obtained from Sigma Aldrich (St. Louis, MO). LN229-luc cells were generated by stable transfection of luciferase-containing lentivirus (EF1a-ffLuc2-eGFP) into naïve U251 cells. Twenty-four hours later, the transfected cells were subjected to puromycin (Sigma) at 1 μg/mL for 7 days. One week after selection, the surviving clones were expanded and followed by extraction of total protein for standard western blotting analysis.

U2OS cells obtained from ATCC were transfected with plasmid containing RNaseH1 construct mutated at D210N (Addgene #111904) and WKKD (Addgene #111905). Stable monoclonal cells were selected with hygromycin.

Renal cell carcinoma cell lines ACHN and 786-O were obtained from ATCC. UM-RC-2 cells were purchased from Sigma, while RCC4 was a gift from Eric Jonasch (MD Anderson). B16-F10 cells were purchased from ATCC. All cells were cultured in Dulbecco's Modified Eagle Medium (DMEM; Gibco) supplemented with 10% fetal bovine serum (FBS; Gibco) and 1% penicillin and streptomycin (Gibco). Small interfering RNA (siRNA) targeting SELH (IDT) were transfected into cells for 48 hours using Lipofectamine RNAiMAX reagent (Invitrogen).

SELH knockout LN229 cell lines and B16 were generated using the CRISPR/Cas9 technique described by Shalem et al., Science 343: 84 (2014). The gDNA for targeting SELH was designed as follows: Oligo 1, 5′-GCCTTACGCTTCCTCCCGCG-3′; Oligo 2, 5′-CTCGGCTACGGCGACCACCG-3′; the gDNA for targeting mouse SELH was designed as follows: Oligo 1, 5′-GTAAGGCGGGGGCCGCGCCTA-3′; Oligo 2, 5′-GCGCCTTACGCTTTCTTCCGT-3′, and subcloned into Cas9 carrying vector (pX330). The two resultant plasmids and puromycin-expressing vector (pPGK-puro) were co-transfected at a ratio of 1:1:1 into LN229 or B16 cells using Lipofectamine 2000 (Invitrogen). Twenty-four hours later, the transfected cells were subjected to puromycin (Sigma) at 1 μg/mL for 7 days. One week after selection, the surviving clones were expanded, followed by extraction of total protein for standard western blotting analysis.

Cell viability assay: Cell viability was measured by Cell Counting Kit-8 (CCK-8, Dojindo Molecular Technologies, Tokyo, Japan). Cells were seeded in 96-well plates at a density of 3×10³ cells/well and cultured for 24 hours before treatment with ISP I at different concentration. After treatment for a gradient ISP I concentration from 0 to 20 μg/ml for 48 hours, while control groups were only treated in PBS solution, 10 μl of CCK-8 solution were added to each well and cells were incubated for an additional 2 hours. The absorbance in each well at a wavelength of OD450 was detected by the Synergy H1 microplate reader, a product of BioTek (Winooski, VT USA).

Cell apoptosis and cell cycle: Cells (2×10⁵) were plated in 6-well plates and treated with different concentrations of ISP I. Then the cells were harvested and washed three times with PBS. Cells were resuspended in 100 μl binding buffer and were incubated with 5 μl APC-conjugated Annexin V working solution, a product of BD bioscience (Franklin Lakes, NJ USA), and 1 μl propidium iodide (PI), a product of Invitrogen, for 15 min at room temperature with protection from light. Data acquisition and quantification were processed with BD LSRFortessa flow cytometer, using FlowJo software (Ashland, OR USA).

For monitoring cell-cycle arrest of ISP I-treated cells, Click-iT EdU Flow Cytometry Assay Kits were used, a product of ThermoFisher Scientific. Cells were co-cultured with EdU at a concentration of 10 μM for 1 hour. After fixation and permeabilization, EdU-positive cells were labeled with Alexa Fluor 647 fluorescein. DAPI also was used for measuring total DNA content to identify differences in cell cycle phases. Data were collected by means of a BD LSRFortessa flow cytometer using FlowJo software. Cells that are positive with EdU and DAPI were in S-Phase of the cell cycle.

Drug Affinity Responsive Target Stability (DARTS) Assay

DARTS assay data were taken to identify the target of ISP I in vitro. For this assay, the invenots used the protocol published by Lomenick et al., Proc. Nat'l Acad. Sci. USA 106: 21984 (2009). Briefly, LN229 cells were lysed with M-PER (Pierce) supplemented with protease and phosphatase inhibitors. After centrifugation at 14,000 rpm for 15 min, lysates were diluted to the same final volume and protein concentration with M-PER and were dissolved in TNC buffer [50 mM Tris-HCl (pH 8.0), 50 mM NaCl, 10 mM CaCl₂]. All steps were performed on ice or at 4° C. to help prevent premature protein degradation. After incubation of the protein sample with ISP I (40 μg/mL) or DMSO as a control at room temperature for one hour, each sample then was proteolyzed with 2 μL 1:100 Pronase at room temperature for 30 min. To stop proteolysis, 3 μL cold 20×Protease inhibitor were added to each sample, mixed well, and placed on ice. The digested peptide was filtered through a Vivacon 500 10K spin column, was precipitated using acetone, and then was digested with trypsin as previously described. Peptides were analyzed by LC/MS/MS on a Thermo LTQ-Orbitrap mass spectrometer with an Eksigent LC pump. For quantitative comparison of protein and peptide abundances, MS spectra were analyzed by means of the differential workflow of Rosetta Elucidator (Rosetta Inpharmatics).

Cellular Thermal Shift Assay (CETSA)

CETSA experiments were performed to determine the ISP I-related ligand induced shifts. Intact and live cells in a 10-cm dish were harvested, washed and resuspended in PBS with protease inhibitor cocktail. Protein of the cells was extracted by freeze-thaw more than three times, using liquid nitrogen. The supernatant was centrifuged at 20,000 g for 20 min at 4° C. Then the samples were aliquoted to several groups incubated with different concentrations of ISP I for 1 hour. After that, samples were aliquoted into PCR tubes and thereafter heated at gradient temperature ranging from 40° C. to 80° C. for 3 min. Then the samples were centrifuged again and resolved using 4-12% SDS-PAGE followed by western blot experiments.

Surface Plasmon Resonance (SPR) Assay

The affinities constant (KD) and kinetics (ka and kd) of ISP I binding to SELH (Sec44→Cys44) were assayed using Biacore 8K (GE Healthcare, Sweden), all at 25° C. The stock solution 10×PBS-P+(with 0.5% P20), provided by GE, was used to prepare running buffers, 4-point solvent correction and samples for binding in 5% DMSO. The purified active SELH (Sec44→Cys44) was diluted by 10 mM sodium acetate solution at pH 5.5, resulting in a protein concentration of 50 μg/mL. Coupling conditions were determined by protein isoelectric points. The diluted protein was immobilized on the surface of a CM5 sensor chip via the primary amine group, employing a standard Amine Coupling Kit, and the target immobilization level was 7000 response units (RUs).

To determine the binding affinity between ISP I and SELH (Sec44→Cys44), a series of ISP I dilutions was analyzed by single-cycle kinetics. As the analyte, a concentration gradient of ISP I was freshly prepared in PBS-P+ running buffer (with 5% DMSO), with at least five concentrations (31.25, 62.5, 125, 250, 500 μM). The ISP I at various gradient concentrations and one zero concentration (running buffer) flowed over the immobilized SELH, with 120 s for binding, followed by disassociation for 120 s, and the obtained response units (RUs) were recorded. The RU values were collected, and the binding affinity data were calculated by kinetic models (1:1 interaction) within Biacore 8K Evaluation Software. The equilibrium dissociation constant (KD) was calculated to evaluate the ability of ISP I to interact with SELH.

Co-Immunoprecipitation

For co-immunoprecipitation analysis, LN229 wild-type cells, LN229 cells treated with 10 μg/mL of ISP I, and LN229 cells transfected with SELH siRNA for 24 hours were harvested, and total protein was prepared from cells using Dynabeads Co-Immunoprecipitation Kit (ThermoFisher) according to the manufacturer's protocol. Immunoblotting then was performed using anti-SELH antibody.

ChIP Assay

ChIP assays were performed using a SimpleCHIP Enzymatic Chromatin IP Kit (Magnetic beads) following the manufacturer's instructions (Cell Signaling Technology; catalog 9003). Cross-linked protein-DNA complexes were precipitated by incubating with rabbit anti-POL1A (CST; 24799s) or rabbit IgG (negative control) overnight and then with magnetic beads for 2 hours. Purified DNA fragments, including HIF2a and ER-binding elements, were analyzed quantitatively by real-time PCR with primers against the rDNA promoter and gene body, following the standard-curve method. The standard curves were created by serial dilution of 2% input chromatin DNA. The values of chromatin DNA precipitated by POL1 antibody were normalized to those precipitated by normal rabbit IgG, which is arbitrarily defined as 1. The primer sequences are described by Frankowski et al., Science translational medicine 10: eaap8370 (2018).

Xenograft and Lung Melanoma Metastasis Mouse Model

Mice experiments were approved by the National Institute of Neurological Disorders and Stroke (NINDS) and National Cancer Institute (NCI) Animal Use and Care Committees. For intracranial xenografts establishment, NOD-Prkde^{scid}Il2rg^{tmiWjl} (NSG) mice (6-8 weeks old from the NCI-Frederick animal facility) were inoculated intracranially with 100,000 LN229-luc cells suspended in 2 μL Hank's Balanced Salt Solution (HBSS), a product of Crystalgen (Commack, NY USA). After one week, luciferin signals were detected to confirm the survival of tumor cells in mice. The mice were assigned to the indicated groups according to the signal intensity, in order to keep the baseline balanced. ISP I was injected intraperitoneally daily at a dose of 66 mg/kg body weight for 24 days. The mice in control group were injected with the same volume of corn oil or saline. The viability of tumors was monitored every four days. Survival end point for all animal studies was defined as when any of the following criteria were reached: 1) a loss of more than 15% of body weight, 2) protruded skull, 3) head tile, 4) hunched posture, 5) ataxia, 6) rough hair coat, or 7) impaired mobility.

For subcutaneous xenografts, NSG mice (6-8 weeks old) from the NCI-Frederick animal facility and Jackson Lab (Bar Harbor, ME USA) were injected with 5×10⁶ to 1×10⁷ cells subcutaneously in the flank. One week later, the tumor-bearing mice were randomly assigned to different groups and made the tumor volume baseline equivalent and treated with normal saline or Zenomycin (35 mg/kg) everyday intraperitoneally. Tumors were measured using a caliper, and volume was calculated.

For metastasis study, C57BL/6 mice (4-5 weeks) from Charles River Laboratories (Wilmington, MA USA) were assigned randomly to one or another of two groups, 9 animals per group. B16-F10 mouse skin melanoma cells (2×10⁵) were resuspended in 100 μl saline and injected through tail vein. After treatment with ISP I at 35 mg/kg for 12 days, all mice were euthanized, and the lungs were examined for counting black metastasis dots.

### STATISTICS

Data were presented as the mean and standard deviation (SD) or standard error of the mean (SEM), as indicated. Other variables were analyzed using two-way ANOVA or unpaired Student's t-test, as appropriate. Statistical analyses were performed using GraphPad Prism 6, a product of GraphPad Software (San Diego, CA USA). A p<0.05 was considered as statistically significant.

The above descriptions are merely preferred embodiments of the present disclosure, rather than a limitation of any form to the present disclosure. Although the present disclosure has been disclosed above in the preferred embodiments, the embodiments are not used for defining the present disclosure. Changes or modifications made by those skilled in the art by virtue of the above technical contents without departing from the scope of the technical solutions of the present disclosure are equivalent embodiments with equivalent changes. However, simple improvements, equivalent changes and modifications made to the above embodiments according to the technical essence of the present disclosure without departing from the contents of the technical solutions of the present disclosure shall belong to the scope of the solutions in the present disclosure.

1-3. (canceled)

4. The method according to claim 5, wherein the selenoprotein interacts with an isovaleryl, and/or an isopentenyl, and/or an isopentenyl-like group of the drug.

5. Method for screening a drug for treating and/or preventing tumors or a drug for preventing tumor metastasis, comprising:
screening the drug by taking selenoprotein as a drug action target.

6. The method according to claim 5, comprising the following steps of:
(1) interacting a candidate drug with the selenoprotein; and
(2) screening the drug for treating and/or preventing tumors or the drug for preventing tumor metastasis according to an affinity between the candidate drug and the selenoprotein;
wherein the candidate drug with a high affinity with the selenoprotein is taken as a candidate primarily screened drug.

7. The method according to claim 6, wherein the candidate primarily screened drug includes a compound with an isopentenyl structure, a compound with an isopentenyl-like structure, a compound with an isovaleryl structure, a macrolide compound and a cyclic peptide compound.

8. The method according to claim 7, wherein the candidate primarily screened drug includes a coumarin compound or a triterpenoid compound or a flavonoid compound or a macrolide compound or a shikonin compound with the isopentenyl structure, and/or the isovaleryl structure, and/or the isopentenyl-like structure.

9. The method according to claim 5, wherein the tumors include solid tumors and non-solid tumors.

10. The method according to claim 6, further comprising:
carrying out an in-vitro test on the candidate primarily screened drug, and further screening out the drug with an inhibiting effect on tumor cells and/or a preventing effect on tumor metastasis.

11. The method according to claim 5, wherein the selenoprotein is human selenoprotein.

12. The method according to claim 5, wherein the selenoprotein is selenoprotein H.

13. The method according to claim 8, wherein the coumarin compound and the triterpenoid compound include aurapten, iso-imperatorin, protopanoxadiol, decursin, osthol, notoginsenoside R1 and shionon.

14. The method according to claim 8, wherein shikonin compound includes acetyl shikonin, anthraquinone, isoxanthohunol, α-mangostin, morusin and shikonin.

15. The method according to claim 7, wherein the macrolide compound and cyclic peptide compound include carrimycin, isovalerylspiramycin I, isovalerylspiramycin II, isovalerylspiramycin III, spiramycin, carbomycin, azithromycin, erythromycin and thiostrepton.

16. The method according to claim 9, wherein the solid tumors include benign solid tumors and malignant solid tumors, and the non-solid tumors include lymphoma or leukemia.

17. The method according to claim 16, wherein the malignant solid tumors include breast cancer, liver cancer, lung cancer, kidney cancer, brain tumor, cervical cancer, prostate cancer, lymph cancer, pancreatic cancer, esophageal cancer, gastric cancer, colon cancer, thyroid cancer, bladder cancer or malignant skin tumor.

18. The method according to claim 17, wherein the malignant skin tumor includes melanoma.
